SEQUENCE LISTING (26 sequences)

SEQ ID NO: 1 - DNA, 3750 bp, Homo sapiens
  source 1..3750: mol_type = genomic DNA
atgggcctaaggcctcccaccccatccccctcagggggctcctgctcaggttccttgccccctccttcccgctgccagcctctccgccgtcgctgctcttcctgctgctttccgggggaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctccatcgaccttcagtccttcatggctcgaggcctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtga

SEQ ID NO: 2 - DNA, 7494 bp, synthetic construct
  source 1..7494: mol_type = other DNA
ggccttaggcctcccaccccatctccctcaggaggctcctgttcagggtccttgccacctccttctcgctgtcagccgctccgcagacgctgttcttcctgctgctttcctggggaataccacttgggtcgctcgaggaggaagagtgttccaggaggaaaacagtacagcatggaaggcgctcctgctgcgcctttccggccctctcaaggcttcctgtctcgaagactaaaaagctccatcaaacgaacgaaatcacaacccaaacttgacagaaccagcagctttcgccagattctgcctagattccgaagtgctgaccatgatcgggcccggctgatgcaaagctttaaagagtcacattctcatgagtccttgctgagtccttctagtgcagctgaagcattggagctcaacttggatgaagattccattataaagccagtgcacagctccatcctgggccaggagttctgttttgaggtaacaacttcatcaggaactaaatgctttgcctgtcggtctgcggccgaaagagataaatggattgaaaatctgcagcgggcagtgaagcccaacaaagacaacagccgccgggtagacaatgtgctaaaactgtggataatagaggccagagagctgccccccaagaagcggtactactgtgaactctgcctggatgacatgctgtatgcacgcactacctccaagccccgctctgcctctggggacaccgttttttggggcgaacacttcgagtttaacaatcttccggctgtccgtgcccttcggttacatctttaccgtgattcagataaaaagcgcaaaaaagacaaggcaggctatgtcggcctggtgactgtgccagtggcgaccctggctgggaggcacttcactgaacagtggtaccctgtaaccctgccgacaggcagtgggggaagtggaggtatgggatctggaggaggagggggctctggtggtggatcaggtggcaagggcaaaggaggctgcccggctgtgaggctgaaagcacgttaccaaacaatgagcattttgcccatggagctatataaagagtttgcagagtatgtcaccaatcattatcggatgttatgtgcagtgttggagcccgccctgaatgtcaaaggcaaggaggaagttgccagtgcactagttcacatcctgcaaagtacaggcaaggccaaggacttcctttcagatatggcgatgtctgaggtggacagattcatggaacgggaacacctcatattcagagagaatacgcttgccactaaagccatagaagagtatatgagactgattgggcaaaaatacctcaaggatgccattggagaattcatacgtgctctgtatgaatctgaggaaaattgtgaggttgatcctatcaagtgcacagcatccagtttggcagaacaccaggccaatctgcgaatgtgctgtgaattggcgttatgcaaagtggtcaactcccactgcgtgttcccgagggagctgaaggaggtgtttgcttcgtggcggctgcgctgtgcagaacgaggcagggaggacatcgcagataggcttatcagcgcctcactcttcttaagattcctctgcccagcgattatgagtcccagtctctttgggcttatgcaagagtacccagatgagcagacctcacgaaccctcaccctcattgccaaggtcatccagaacctggccaacttttctaaatttacctcaaaggaagattttttaggcttcatgaatgagtttttagaactggaatggggttccatgcagcagtttttgtatgagatctccaatctggacacgctaactaatagcagttcttttgaggggtacatcgacttgggccgagagctctccactctgcatgccctactttgggaagtgcttccccagctcagcaaggaagccctcctgaagctgggtccattacccaggcttctcaacgacatcagcacagctctgaggaatcccaacattcaaaggcagccaagccgccaaagtgagcggcctagacctcagcctgtggtcctgagagggccttcggctgagatgcagggctacatgatgagagacctcaacagctccatcgaccttcagtccttcatggctcgaggcctcaatagctctatggacatggctcgcctcccctctccaaccaaggaaaagccacccccaccaccgcctggtggtggtaaagacttattctatgtaagcagaccacccctggcccgttcctcaccagcatactgcacgtctagctcggacatcacagaaccagagcagaagatgctgagtgttaacaagagtgtgtcaatgctggacttacagggggatgggcctggtggcagactcaactctagcagtgtttcgaatctggcggctgtaggggatctgctgcactcaagccaggcctcgctgacagcagccttggggctacggcctgcacctgctggaagactctcccaggggagtggctcatccattacggcggctggcatgagacttagccagatgggtgttactacagacggagtccctgcccagcaactgcgaatacccctctccttccagaaccctctcttccatatggctgctgatgggccaggccctccaggtggacatggaggaggaggtggccatggcccaccttcctcccatcatcaccatcaccaccatcaccaccacagaggaggagagccccctggggatacctttgccccattccatggctatagcaagagtgaggacctctcttccggggtgcccaagccccctgctgcctccatccttcatagccacagctacagtgatgaatttggaccctctggcactgatttcacccgtcgccaactttcactccaggacaacctgcagcacatgctttcccctcctcagatcaccattggtcctcagaggccagcaccctcaggacctggaggtgggtctggcggtggctctggtggtggaggaggaggccagcctcctccattgcagaggggcaagtctcagcagttgacagtcagcgcagcccagaaaccgagaccatcaagcgggaatctattgcaatccccagagccgagttatggccccgcccgtccaagacaacagagcctcagcaaggaaggcagcattggaggcagtggaggcagcggtggtggaggaggtggagggctgaagccctccatcactaaacagcattctcagactccatccacattgaaccccacaatgccagcttctgagcgcacagtggcctgggtctccaacatgcctcacctgtctgctgacattgagagtgcccacatcgagcgggaagaatacaagctcaaggagtactcaaaatcgatggatgagagccggctggatagggtgaaggagtacgaagaggagattcattcactgaaagagagattacatatgtccaatcgtaaactggaagaatatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtgaggccttaggcctcccaccccatctccctcaggaggctcctgttcagggtccttgccacctccttctcgctgtcagccgctccgcagacgctgttcttcctgctgctttcctggggaataccacttgggtcgctcgaggaggaagagtgttccaggaggaaaacagtacagcatggaaggcgctcctgctgcgcctttccggccctctcaaggcttcctgtctcgaagactaaaaagctccatcaaacgaacgaaatcacaacccaaacttgacagaaccagcagctttcgccagattctgcctagattccgaagtgctgaccatgatcgggcccggctgatgcaaagctttaaagagtcacattctcatgagtccttgctgagtccttctagtgcagctgaagcattggagctcaacttggatgaagattccattataaagccagtgcacagctccatcctgggccaggagttctgttttgaggtaacaacttcatcaggaactaaatgctttgcctgtcggtctgcggccgaaagagataaatggattgaaaatctgcagcgggcagtgaagcccaacaaagacaacagccgccgggtagacaatgtgctaaaactgtggataatagaggccagagagctgccccccaagaagcggtactactgtgaactctgcctggatgacatgctgtatgcacgcactacctccaagccccgctctgcctctggggacaccgttttttggggcgaacacttcgagtttaacaatcttccggctgtccgtgcccttcggttacatctttaccgtgattcagataaaaagcgcaaaaaagacaaggcaggctatgtcggcctggtgactgtgccagtggcgaccctggctgggaggcacttcactgaacagtggtaccctgtaaccctgccgacaggcagtgggggaagtggaggtatgggatctggaggaggagggggctctggtggtggatcaggtggcaagggcaaaggaggctgcccggctgtgaggctgaaagcacgttaccaaacaatgagcattttgcccatggagctatataaagagtttgcagagtatgtcaccaatcattatcggatgttatgtgcagtgttggagcccgccctgaatgtcaaaggcaaggaggaagttgccagtgcactagttcacatcctgcaaagtacaggcaaggccaaggacttcctttcagatatggcgatgtctgaggtggacagattcatggaacgggaacacctcatattcagagagaatacgcttgccactaaagccatagaagagtatatgagactgattgggcaaaaatacctcaaggatgccattggagaattcatacgtgctctgtatgaatctgaggaaaattgtgaggttgatcctatcaagtgcacagcatccagtttggcagaacaccaggccaatctgcgaatgtgctgtgaattggcgttatgcaaagtggtcaactcccactgcgtgttcccgagggagctgaaggaggtgtttgcttcgtggcggctgcgctgtgcagaacgaggcagggaggacatcgcagataggcttatcagcgcctcactcttcttaagattcctctgcccagcgattatgagtcccagtctctttgggcttatgcaagagtacccagatgagcagacctcacgaaccctcaccctcattgccaaggtcatccagaacctggccaacttttctaaatttacctcaaaggaagattttttaggcttcatgaatgagtttttagaactggaatggggttccatgcagcagtttttgtatgagatctccaatctggacacgctaactaatagcagttcttttgaggggtacatcgacttgggccgagagctctccactctgcatgccctactttgggaagtgcttccccagctcagcaaggaagccctcctgaagctgggtccattacccaggcttctcaacgacatcagcacagctctgaggaatcccaacattcaaaggcagccaagccgccaaagtgagcggcctagacctcagcctgtggtcctgagagggccttcggctgagatgcagggctacatgatgagagacctcaacagctccatcgaccttcagtccttcatggctcgaggcctcaatagctctatggacatggctcgcctcccctctccaaccaaggaaaagccacccccaccaccgcctggtggtggtaaagacttattctatgtaagcagaccacccctggcccgttcctcaccagcatactgcacgtctagctcggacatcacagaaccagagcagaagatgctgagtgttaacaagagtgtgtcaatgctggacttacagggggatgggcctggtggcagactcaactctagcagtgtttcgaatctggcggctgtaggggatctgctgcactcaagccaggcctcgctgacagcagccttggggctacggcctgcacctgctggaagactctcccaggggagtggctcatccattacggcggctggcatgagacttagccagatgggtgttactacagacggagtccctgcccagcaactgcgaatacccctctccttccagaaccctctcttccatatggctgctgatgggccaggccctccaggtggacatggaggaggaggtggccatggcccaccttcctcccatcatcaccatcaccaccatcaccaccacagaggaggagagccccctggggatacctttgccccattccatggctatagcaagagtgaggacctctcttccggggtgcccaagccccctgctgcctccatccttcatagccacagctacagtgatgaatttggaccctctggcactgatttcacccgtcgccaactttcactccaggacaacctgcagcacatgctttcccctcctcagatcaccattggtcctcagaggccagcaccctcaggacctggaggtgggtctggcggtggctctggtggtggaggaggaggccagcctcctccattgcagaggggcaagtctcagcagttgacagtcagcgcagcccagaaaccgagaccatcaagcgggaatctattgcaatccccagagccgagttatggccccgcccgtccaagacaacagagcctcagcaaggaaggcagcattggaggcagtggaggcagcggtggtggaggaggtggagggctgaagccctccatcactaaacagcattctcagactccatccacattgaaccccacaatgccagcttctgagcgcacagtggcctgggtctccaacatgcctcacctgtctgctgacattgagagtgcccacatcgagcgggaagaatacaagctcaaggagtactcaaaatcgatggatgagagccggctggatagggtgaaggagtacgaagaggagattcattcactgaaagagagattacatatgtccaatcgtaaactggaagaatatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtga

SEQ ID NO: 3 - DNA, 3852 bp, synthetic construct
  source 1..3852: mol_type = other DNA
ggtcttaggcctcccaccccatctccctcaggaggctcctgttcagggtccttgccacctccttctcgctgtcagccgctccgcagacgctgttcttcctgctgctttcctggggaataccacttgggtcgctcgaggaggaagagtgttccaggaggaaaacagtacagcatggaaggcgctcctgctgcgcctttccggccctctcaaggcttcctgtctcgaagactaaaaagctccatcaaacgaacgaaatcacaacccaaacttgacagaaccagcagctttcgccagattctgcctagattccgaagtgctgaccatgatcgggcccggctgatgcaaagctttaaagagtcacattctcatgagtccttgctgagtccttctagtgcagctgaagcattggagctcaacttggatgaagattccattataaagccagtgcacagctccatcctgggccaggagttctgttttgaggtaacaacttcatcaggaactaaatgctttgcctgtcggtctgcggccgaaagagataaatggattgaaaatctgcagcgggcagtgaagcccaacaaagacaacagccgccgggtagacaatgtgctaaaactgtggataatagaggccagagagctgccccccaagaagcggtactactgtgaactctgcctggatgacatgctgtatgcacgcactacctccaagccccgctctgcctctggggacaccgttttttggggcgaacacttcgagtttaacaatcttccggctgtccgtgcccttcggttacatctttaccgtgattcagataaaaagcgcaaaaaagacaaggcaggctatgtcggcctggtgactgtgccagtggcgaccctggctgggaggcacttcactgaacagtggtaccctgtaaccctgccgacaggcagtgggggaagtggaggtatgggatctggaggaggagggggctctggtggtggatcaggtggcaagggcaaaggaggctgcccggctgtgaggctgaaagcacgttaccaaacaatgagcattttgcccatggagctatataaagagtttgcagagtatgtcaccaatcattatcggatgttatgtgcagtgttggagcccgccctgaatgtcaaaggcaaggaggaagttgccagtgcactagttcacatcctgcaaagtacaggcaaggccaaggacttcctttcagatatggcgatgtctgaggtggacagattcatggaacgggaacacctcatattcagagagaatacgcttgccactaaagccatagaagagtatatgagactgattgggcaaaaatacctcaaggatgccattggagaattcatacgtgctctgtatgaatctgaggaaaattgtgaggttgatcctatcaagtgcacagcatccagtttggcagaacaccaggccaatctgcgaatgtgctgtgaattggcgttatgcaaagtggtcaactcccactgcgtgttcccgagggagctgaaggaggtgtttgcttcgtggcggctgcgctgtgcagaacgaggcagggaggacatcgcagataggcttatcagcgcctcactcttcttaagattcctctgcccagcgattatgagtcccagtctctttgggcttatgcaagagtacccagatgagcagacctcacgaaccctcaccctcattgccaaggtcatccagaacctggccaacttttctaaatttacctcaaaggaagattttttaggcttcatgaatgagtttttagaactggaatggggttccatgcagcagtttttgtatgagatctccaatctggacacgctaactaatagcagttcttttgaggggtacatcgacttgggccgagagctctccactctgcatgccctactttgggaagtgcttccccagctcagcaaggaagccctcctgaagctgggtccattacccaggcttctcaacgacatcagcacagctctgaggaatcccaacattcaaaggcagccaagccgccaaagtgagcggcctagacctcagcctgtggtcctgagagggccttcggctgagatgcagggctacatgatgagagacctcaacagctccatcgaccttcagtccttcatggctcgaggcctcaatagctctatggacatggctcgcctcccctctccaaccaaggaaaagccacccccaccaccgcctggtggtggtaaagacttattctatgtaagcagaccacccctggcccgttcctcaccagcatactgcacgtctagctcggacatcacagaaccagagcagaagatgctgagtgttaacaagagtgtgtcaatgctggacttacagggggatgggcctggtggcagactcaactctagcagtgtttcgaatctggcggctgtaggggatctgctgcactcaagccaggcctcgctgacagcagccttggggctacggcctgcacctgctggaagactctcccaggggagtggctcatccattacggcggctggcatgagacttagccagatgggtgttactacagacggagtccctgcccagcaactgcgaatacccctctccttccagaaccctctcttccatatggctgctgatgggccaggccctccaggtggacatggaggaggaggtggccatggcccaccttcctcccatcatcaccatcaccaccatcaccaccacagaggaggagagccccctggggatacctttgccccattccatggctatagcaagagtgaggacctctcttccggggtgcccaagccccctgctgcctccatccttcatagccacagctacagtgatgaatttggaccctctggcactgatttcacccgtcgccaactttcactccaggacaacctgcagcacatgctttcccctcctcagatcaccattggtcctcagaggccagcaccctcaggacctggaggtgggtctggcggtggctctggtggtggaggaggaggccagcctcctccattgcagaggggcaagtctcagcagttgacagtcagcgcagcccagaaaccgagaccatcaagcgggaatctattgcaatccccagagccgagttatggccccgcccgtccaagacaacagagcctcagcaaggaaggcagcattggaggcagtggaggcagcggtggtggaggaggtggagggctgaagccctccatcactaaacagcattctcagactccatccacattgaaccccacaatgccagcttctgagcgcacagtggcctgggtctccaacatgcctcacctgtctgctgacattgagagtgcccacatcgagcgggaagaatacaagctcaaggagtactcaaaatcgatggatgagagccggctggatagggtgaaggaatacgaagaggagattcattcactgaaagagagattacatatgtccaatcgtaaactggaagaatatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcaggagaggcaacttcctcccttgggaccaacaaacccgcgggtgaccctggcaccaccttggaatggcctcgccccccctgctccaccaccccctcccagactgcagatcactgagaacggtgaattccgtaacaccgcagaccactag

SEQ ID NO: 4 - DNA, 3678 bp, synthetic construct
  source 1..3678: mol_type = other DNA
ggattgaggcctcccaccccatctccctcaggaggctcctgttcagggtccttgccacctccttctcgctgtcagccgctccgcagacgctgttcttcctgctgctttcctggggaataccacttgggtcgctcgaggaggaagagtgttccaggaggaaaacagtacagcatggaaggcgctcctgctgcgcctttccggccctctcaaggcttcctgtctcgaagactaaaaagctccatcaaacgaacgaaatcacaacccaaacttgacagaaccagcagctttcgccagattctgcctagattccgaagtgctgaccatgatcgggcccggctgatgcaaagctttaaagagtcacattctcatgagtccttgctgagtccttctagtgcagctgaagcattggagctcaacttggatgaagattccattataaagccagtgcacagctccatcctgggccaggagttctgttttgaggtaacaacttcatcaggaactaaatgctttgcctgtcggtctgcggccgaaagagataaatggattgaaaatctgcagcgggcagtgaagcccaacaaagacaacagccgccgggtagacaatgtgctaaaactgtggataatagaggccagagagctgccccccaagaagcggtactactgtgaactctgcctggatgacatgctgtatgcacgcactacctccaagccccgctctgcctctggggacaccgttttttggggcgaacacttcgagtttaacaatcttccggctgtccgtgcccttcggttacatctttaccgtgattcagataaaaagcgcaaaaaagacaaggcaggctatgtcggcctggtgactgtgccagtggcgaccctggctgggaggcacttcactgaacagtggtaccctgtaaccctgccgacaggcagtgggggaagtggaggtatgggatctggaggaggagggggctctggtggtggatcaggtggcaagggcaaaggaggctgcccggctgtgaggctgaaagcacgttaccaaacaatgagcattttgcccatggagctatataaagagtttgcagagtatgtcaccaatcattatcggatgttatgtgcagtgttggagcccgccctgaatgtcaaaggcaaggaggaagttgccagtgcactagttcacatcctgcaaagtacaggcaaggccaaggacttcctttcagatatggcgatgtctgaggtggacagattcatggaacgggaacacctcatattcagagagaatacgcttgccactaaagccatagaagagtatatgagactgattgggcaaaaatacctcaaggatgccattggagaattcatacgtgctctgtatgaatctgaggaaaattgtgaggttgatcctatcaagtgcacagcatccagtttggcagaacaccaggccaatctgcgaatgtgctgtgaattggcgttatgcaaagtggtcaactcccactgcgtgttcccgagggagctgaaggaggtgtttgcttcgtggcggctgcgctgtgcagaacgaggcagggaggacatcgcagataggcttatcagcgcctcactcttcttaagattcctctgcccagcgattatgagtcccagtctctttgggcttatgcaagagtacccagatgagcagacctcacgaaccctcaccctcattgccaaggtcatccagaacctggccaacttttctaaatttacctcaaaggaagattttttaggcttcatgaatgagtttttagaactggaatggggttccatgcagcagtttttgtatgagatctccaatctggacacgctaactaatagcagttcttttgaggggtacatcgacttgggccgagagctctccactctgcatgccctactttgggaagtgcttccccagctcagcaaggaagccctcctgaagctgggtccattacccaggcttctcaacgacatcagcacagctctgaggaatcccaacattcaaaggcagccaagccgccaaagtgagcggcctagacctcagcctgtggtcctgagagggccttcggctgagatgcagggctacatgatgagagacctcaacagctccatcgaccttcagtccttcatggctcgaggcctcaatagctctatggacatggctcgcctcccctctccaaccaaggaaaagccacccccaccaccgcctggtggtggtaaagacttattctatgtaagcagaccacccctggcccgttcctcaccagcatactgcacgtctagctcggacatcacagaaccagagcagaagatgctgagtgttaacaagagtgtgtcaatgctggacttacagggggatgggcctggtggcagactcaactctagcagtgtttcgaatctggcggctgtaggggatctgctgcactcaagccaggcctcgctgacagcagccttggggctacggcctgcacctgctggaagactctcccaggggagtggctcatccattacggcggctggcatgagacttagccagatgggtgttactacagacggagtccctgcccagcaactgcgaatacccctctccttccagaaccctctcttccatatggctgctgatgggccaggccctccaggtggacatggaggaggaggtggccatggcccaccttcctcccatcatcaccatcaccaccatcaccaccacagaggaggagagccccctggggatacctttgccccattccatggctatagcaagagtgaggacctctcttccggggtgcccaagccccctgctgcctccatccttcatagccacagctacagtgatgaatttggaccctctggcactgatttcacccgtcgccaactttcactccaggacaacctgcagcacatgctttcccctcctcagatcaccattggtcctcagaggccagcaccctcaggacctggaggtgggtctggcggtggctctggtggtggaggaggaggccagcctcctccattgcagaggggcaagtctcagcagttgacagtcagcgcagcccagaaaccgagaccatcaagcgggaatctattgcaatccccagagccgagttatggccccgcccgtccaagacaacagagcctcagcaaggaaggcagcattggaggcagtggaggcagcggtggtggaggaggtggagggctgaagccctccatcactaaacagcattctcagactccatccacattgaaccccacaatgccagcttctgagcgcacagtggcctgggtctccaacatgcctcacctgtctgctgacattgagagtgcccacatcgagcgggaagaatacaagctcaaggagtactcaaaatcgatggatgagagccggctggatagggtgaaggagtacgaagaagagatccactcactgaaagagagactccacatgtccaacagaaagttggaagagtatgagcggaggctactgtctcaggaagaacaaacctcgaaaatattaatgcagtaccaggcccgactggaacaaagtgagaagaggctaaggcagcagcaggcagagaaggattctcaaatcaagagcatcattggcagcccgtcccttcaggctgacgctggtggagggggggccgcgcctggaccaccccgccatggctga

SEQ ID NO: 5 - DNA, 3720 bp, synthetic construct
  source 1..3720: mol_type = other DNA
gggttgaggcctcccaccccatctccctcaggaggctcctgttcagggtccttgccacctccttctcgctgtcagccgctccgcagacgctgttcttcctgctgctttcctggggaataccacttgggtcgctcgaggaggaagagtgttccaggaggaaaacagtacagcatggaaggcgctcctgctgcgcctttccggccctctcaaggcttcctgtctcgaagactaaaaagctccatcaaacgaacgaaatcacaacccaaacttgacagaaccagcagctttcgccagattctgcctagattccgaagtgctgaccatgatcgggcccggctgatgcaaagctttaaagagtcacattctcatgagtccttgctgagtccttctagtgcagctgaagcattggagctcaacttggatgaagattccattataaagccagtgcacagctccatcctgggccaggagttctgttttgaggtaacaacttcatcaggaactaaatgctttgcctgtcggtctgcggccgaaagagataaatggattgaaaatctgcagcgggcagtgaagcccaacaaagacaacagccgccgggtagacaatgtgctaaaactgtggataatagaggccagagagctgccccccaagaagcggtactactgtgaactctgcctggatgacatgctgtatgcacgcactacctccaagccccgctctgcctctggggacaccgttttttggggcgaacacttcgagtttaacaatcttccggctgtccgtgcccttcggttacatctttaccgtgattcagataaaaagcgcaaaaaagacaaggcaggctatgtcggcctggtgactgtgccagtggcgaccctggctgggaggcacttcactgaacagtggtaccctgtaaccctgccgacaggcagtgggggaagtggaggtatgggatctggaggaggagggggctctggtggtggatcaggtggcaagggcaaaggaggctgcccggctgtgaggctgaaagcacgttaccaaacaatgagcattttgcccatggagctatataaagagtttgcagagtatgtcaccaatcattatcggatgttatgtgcagtgttggagcccgccctgaatgtcaaaggcaaggaggaagttgccagtgcactagttcacatcctgcaaagtacaggcaaggccaaggacttcctttcagatatggcgatgtctgaggtggacagattcatggaacgggaacacctcatattcagagagaatacgcttgccactaaagccatagaagagtatatgagactgattgggcaaaaatacctcaaggatgccattggagaattcatacgtgctctgtatgaatctgaggaaaattgtgaggttgatcctatcaagtgcacagcatccagtttggcagaacaccaggccaatctgcgaatgtgctgtgaattggcgttatgcaaagtggtcaactcccactgcgtgttcccgagggagctgaaggaggtgtttgcttcgtggcggctgcgctgtgcagaacgaggcagggaggacatcgcagataggcttatcagcgcctcactcttcttaagattcctctgcccagcgattatgagtcccagtctctttgggcttatgcaagagtacccagatgagcagacctcacgaaccctcaccctcattgccaaggtcatccagaacctggccaacttttctaaatttacctcaaaggaagattttttaggcttcatgaatgagtttttagaactggaatggggttccatgcagcagtttttgtatgagatctccaatctggacacgctaactaatagcagttcttttgaggggtacatcgacttgggccgagagctctccactctgcatgccctactttgggaagtgcttccccagctcagcaaggaagccctcctgaagctgggtccattacccaggcttctcaacgacatcagcacagctctgaggaatcccaacattcaaaggcagccaagccgccaaagtgagcggcctagacctcagcctgtggtcctgagagggccttcggctgagatgcagggctacatgatgagagacctcaacagctccatcgaccttcagtccttcatggctcgaggcctcaatagctctatggacatggctcgcctcccctctccaaccaaggaaaagccacccccaccaccgcctggtggtggtaaagacttattctatgtaagcagaccacccctggcccgttcctcaccagcatactgcacgtctagctcggacatcacagaaccagagcagaagatgctgagtgttaacaagagtgtgtcaatgctggacttacagggggatgggcctggtggcagactcaactctagcagtgtttcgaatctggcggctgtaggggatctgctgcactcaagccaggcctcgctgacagcagccttggggctacggcctgcacctgctggaagactctcccaggggagtggctcatccattacggcggctggcatgagacttagccagatgggtgttactacagacggagtccctgcccagcaactgcgaatacccctctccttccagaaccctctcttccatatggctgctgatgggccaggccctccaggtggacatggaggaggaggtggccatggcccaccttcctcccatcatcaccatcaccaccatcaccaccacagaggaggagagccccctggggatacctttgccccattccatggctatagcaagagtgaggacctctcttccggggtgcccaagccccctgctgcctccatccttcatagccacagctacagtgatgaatttggaccctctggcactgatttcacccgtcgccaactttcactccaggacaacctgcagcacatgctttcccctcctcagatcaccattggtcctcagaggccagcaccctcaggacctggaggtgggtctggcggtggctctggtggtggaggaggaggccagcctcctccattgcagaggggcaagtctcagcagttgacagtcagcgcagcccagaaaccgagaccatcaagcgggaatctattgcaatccccagagccgagttatggccccgcccgtccaagacaacagagcctcagcaaggaaggcagcattggaggcagtggaggcagcggtggtggaggaggtggagggctgaagccctccatcactaaacagcattctcagactccatccacattgaaccccacaatgccagcttctgagcgcacagtggcctgggtctccaacatgcctcacctgtctgctgacattgagagtgcccacatcgagcgggaagaatacaagctcaaggagtactcaaaatcgatggatgagagccggctggatagggtgaaggagtacgaagaggaaatccattcactgaaagagagattacatatgtccaatcgtaaactggaagaatatgagcggaggctgctgtcccaggaagaacaaaccagcaaaatcctgatgcagtatcaggcccgactggagcagagtgagaagaggctaaggcagcagcaggcagagaaggattcccagatcaagagcataattggcaggctgatgctggtggaggaggagcttcgccgggaccacccggccatggcagaacctttgccagaacccaagaaaagactgttagatgctcagctcctcatccgatga

SEQ ID NO: 6 - DNA, 3771 bp, synthetic construct
  source 1..3771: mol_type = other DNA
gactacaaggacgatgacgataagggccttaggcctcccaccccatctccctcaggaggctcctgttcagggtccttgccacctccttctcgctgtcagccgctccgcagacgctgttcttcctgctgctttcctggggaataccacttgggtcgctcgaggaggaagagtgttccaggaggaaaacagtacagcatggaaggcgctcctgctgcgcctttccggccctctcaaggcttcctgtctcgaagactaaaaagctccatcaaacgaacgaaatcacaacccaaacttgacagaaccagcagctttcgccagattctgcctagattccgaagtgctgaccatgatcgggcccggctgatgcaaagctttaaagagtcacattctcatgagtccttgctgagtccttctagtgcagctgaagcattggagctcaacttggatgaagattccattataaagccagtgcacagctccatcctgggccaggagttctgttttgaggtaacaacttcatcaggaactaaatgctttgcctgtcggtctgcggccgaaagagataaatggattgaaaatctgcagcgggcagtgaagcccaacaaagacaacagccgccgggtagacaatgtgctaaaactgtggataatagaggccagagagctgccccccaagaagcggtactactgtgaactctgcctggatgacatgctgtatgcacgcactacctccaagccccgctctgcctctggggacaccgttttttggggcgaacacttcgagtttaacaatcttccggctgtccgtgcccttcggttacatctttaccgtgattcagataaaaagcgcaaaaaagacaaggcaggctatgtcggcctggtgactgtgccagtggcgaccctggctgggaggcacttcactgaacagtggtaccctgtaaccctgccgacaggcagtgggggaagtggaggtatgggatctggaggaggagggggctctggtggtggatcaggtggcaagggcaaaggaggctgcccggctgtgaggctgaaagcacgttaccaaacaatgagcattttgcccatggagctatataaagagtttgcagagtatgtcaccaatcattatcggatgttatgtgcagtgttggagcccgccctgaatgtcaaaggcaaggaggaagttgccagtgcactagttcacatcctgcaaagtacaggcaaggccaaggacttcctttcagatatggcgatgtctgaggtggacagattcatggaacgggaacacctcatattcagagagaatacgcttgccactaaagccatagaagagtatatgagactgattgggcaaaaatacctcaaggatgccattggagaattcatacgtgctctgtatgaatctgaggaaaattgtgaggttgatcctatcaagtgcacagcatccagtttggcagaacaccaggccaatctgcgaatgtgctgtgaattggcgttatgcaaagtggtcaactcccactgcgtgttcccgagggagctgaaggaggtgtttgcttcgtggcggctgcgctgtgcagaacgaggcagggaggacatcgcagataggcttatcagcgcctcactcttcttaagattcctctgcccagcgattatgagtcccagtctctttgggcttatgcaagagtacccagatgagcagacctcacgaaccctcaccctcattgccaaggtcatccagaacctggccaacttttctaaatttacctcaaaggaagattttttaggcttcatgaatgagtttttagaactggaatggggttccatgcagcagtttttgtatgagatctccaatctggacacgctaactaatagcagttcttttgaggggtacatcgacttgggccgagagctctccactctgcatgccctactttgggaagtgcttccccagctcagcaaggaagccctcctgaagctgggtccattacccaggcttctcaacgacatcagcacagctctgaggaatcccaacattcaaaggcagccaagccgccaaagtgagcggcctagacctcagcctgtggtcctgagagggccttcggctgagatgcagggctacatgatgagagacctcaacagctccatcgaccttcagtccttcatggctcgaggcctcaatagctctatggacatggctcgcctcccctctccaaccaaggaaaagccacccccaccaccgcctggtggtggtaaagacttattctatgtaagcagaccacccctggcccgttcctcaccagcatactgcacgtctagctcggacatcacagaaccagagcagaagatgctgagtgttaacaagagtgtgtcaatgctggacttacagggggatgggcctggtggcagactcaactctagcagtgtttcgaatctggcggctgtaggggatctgctgcactcaagccaggcctcgctgacagcagccttggggctacggcctgcacctgctggaagactctcccaggggagtggctcatccattacggcggctggcatgagacttagccagatgggtgttactacagacggagtccctgcccagcaactgcgaatacccctctccttccagaaccctctcttccatatggctgctgatgggccaggccctccaggtggacatggaggaggaggtggccatggcccaccttcctcccatcatcaccatcaccaccatcaccaccacagaggaggagagccccctggggatacctttgccccattccatggctatagcaagagtgaggacctctcttccggggtgcccaagccccctgctgcctccatccttcatagccacagctacagtgatgaatttggaccctctggcactgatttcacccgtcgccaactttcactccaggacaacctgcagcacatgctttcccctcctcagatcaccattggtcctcagaggccagcaccctcaggacctggaggtgggtctggcggtggctctggtggtggaggaggaggccagcctcctccattgcagaggggcaagtctcagcagttgacagtcagcgcagcccagaaaccgagaccatcaagcgggaatctattgcaatccccagagccgagttatggccccgcccgtccaagacaacagagcctcagcaaggaaggcagcattggaggcagtggaggcagcggtggtggaggaggtggagggctgaagccctccatcactaaacagcattctcagactccatccacattgaaccccacaatgccagcttctgagcgcacagtggcctgggtctccaacatgcctcacctgtctgctgacattgagagtgcccacatcgagcgggaagaatacaagctcaaggagtactcaaaatcgatggatgagagccggctggatagggtgaaagagtacgaagaggagatacattcactgaaagagagattacatatgtccaatcgtaaactggaagaatatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtga

SEQ ID NO: 7 - protein, 1249 aa, synthetic construct
MGLRPPTPSPSGGSCSGSLPPPSRCQPLRRRCSSCCFPGEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQRGSFPPWVQQTRV

SEQ ID NO: 8 - protein, 1284 aa, synthetic construct
MGLRPPTPSPSGGSCSGSLPPPSRCQPLRRRCSSCCFPGEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQERQLPPLGPTNPRVTLAPPWNGLAPPAPPPPPRLQITENGEFRNTADH

SEQ ID NO: 9 - protein, 1226 aa, synthetic construct
MGLRPPTPSPSGGSCSGSLPPPSRCQPLRRRCSSCCFPGEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGSPSLQADAGGGGAAPGPPRHG

SEQ ID NO: 10 - protein, 1239 aa, synthetic construct
GLRPPTPSPSGGSCSGSLPPPSRCQPLRRRCSSCCFPGEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQLLIR

SEQ ID NO: 11 - protein, 546 aa, synthetic construct
LNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPAGHGGSSGHGPPSSHHHHHHHHHHRGGEPPGDTFAPFHGYSKSEDLSTGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQVEKDSQIKSIIGRLMLVEEELRRDHPAMAEPLPEPKKRLLDAQRGSFPPWVQQTRV

SEQ ID NO: 12 - DNA, 20 bp, synthetic construct
  source 1..20: mol_type = other DNA
ccttatcgtcatcgtccttg

SEQ ID NO: 13 - DNA, 20 bp, synthetic construct
  source 1..20: mol_type = other DNA
ttacggaattcaccgttctc

SEQ ID NO: 14 - DNA, 20 bp, synthetic construct
  source 1..20: mol_type = other DNA
taaggcccttatcgtcatcg

SEQ ID NO: 15 - DNA, 20 bp, synthetic construct
  source 1..20: mol_type = other DNA
agacctttatcgtcatcgtc

SEQ ID NO: 16 - DNA, 20 bp, synthetic construct
  source 1..20: mol_type = other DNA
aatcctttatcgtcatcgtc

SEQ ID NO: 17 - DNA, 20 bp, synthetic construct
  source 1..20: mol_type = other DNA
aaccctttatcgtcatcgtc

SEQ ID NO: 18 - DNA, 20 bp, synthetic construct
  source 1..20: mol_type = other DNA
tatctcctcttcgtactctt

SEQ ID NO: 19 - DNA, 20 bp, synthetic construct
  source 1..20: mol_type = other DNA
aatctcctcttcgtattcct

SEQ ID NO: 20 - DNA, 20 bp, synthetic construct
  source 1..20: mol_type = other DNA
gatctcttcttcgtactcct

SEQ ID NO: 21 - DNA, 20 bp, synthetic construct
  source 1..20: mol_type = other DNA
gatttcctcttcgtactcct

SEQ ID NO: 22 - DNA, 3816 bp, synthetic construct
  source 1..3816: mol_type = other DNA
atggactacaaagaccatgatggtgactataaggaccatgacatagactacaaggacgatgacgataagggcctgaggccacccacaccctccccatcaggtgggtcttgctcagggtccctaccaccaccctctaggtgccagcccctaaggagaaggtgcagctcatgctgcttcccaggtgagtaccacctaggtaggtctaggagaaagtctgtgccagggggtaagcagtactctatggagggtgctcccgctgctcccttcagaccatcccagggtttcctatccaggaggctaaagagctctattaagaggacaaagagccaacccaagctagacaggacatcatctttcaggcagattctgcctaggttcaggtcagctgaccacgacagggccagactaatgcagagcttcaaggagtcccactcacatgagtctctactatcccccagttcagccgcagaggctttggagctcaacctagatgaggactctatcatcaagccagtgcactcaagcatcctaggtcaggagttctgcttcgaggtcacaaccagctcaggcaccaagtgctttgcttgcagatctgctgccgagagggacaagtggattgagaacctacagagggctgtgaagccaaacaaggacaacagcaggagagtggacaatgtgctcaagttgtggatcattgaggctagagagttgcccccaaagaagaggtactactgtgagttgtgcctggacgacatgctctatgctagaaccacaagcaagcccaggtctgcctcaggtgacacagtgttctggggtgagcactttgagttcaacaacctacccgctgtcagggccctaaggttgcacctctacagggactctgacaagaagaggaagaaggacaaggcaggctatgtgggtttggtcacagtgccagtggctacactagctgggaggcacttcactgagcagtggtacccagtgacactgccaacagggtctggtggttcagggggcatggggtctgggggcggtgggggcagtggagggggttctggtggcaaggggaagggtggttgcccagctgttaggcttaaggctaggtaccagacaatgagcattttgcccatggagttgtacaaggagtttgcagagtatgtcaccaaccactacagaatgctgtgtgcagtcttggagccagccctgaatgtcaagggtaaggaggaggttgcctctgctttggtccacatcttgcagtccacaggtaaggccaaggacttcttgtctgacatggcaatgtctgaggttgacaggttcatggagagagagcacctaattttcagggagaacacactggccacaaaggctattgaggagtacatgaggctgattgggcagaagtacctaaaggatgccattggtgagttcatcagggctctctatgagagtgaggagaactgcgaggtggaccccattaagtgcaccgcctcatccctagctgagcaccaggctaacctaaggatgtgctgtgagctagccctatgcaaggtggttaactcccactgcgtgttcccaagggagttgaaggaggtgtttgcatcctggcgactgaggtgcgctgagcgaggcagggaggacatagcagacaggctcatctcggcttccctgttcctgaggttcctgtgccctgctattatgtctccctccctatttgggcttatgcaggagtacccagatgaacagacctctaggaccctcaccctcattgctaaggtcattcagaacctggctaacttctctaagttcacatccaaggaagacttcctaggcttcatgaatgagttcctggagctagagtggggctccatgcagcagttcctatacgagatcagcaacctggacaccctaaccaactcttcaagctttgaggggtacattgacttgggtagggagctctccacactgcatgccctactatgggaggtgctaccacagttgtcaaaggaggccctgctgaagttggggccactccccaggctgctaaatgacattagcacagctctaaggaaccccaacatccagaggcagccaagcaggcagtctgagagaccaaggccccagccagttgttctgagggggcccagtgctgagatgcagggctacatgatgagggacctaaactcctctattgaccttcagtctttcatggctaggggcctcaacagctcaatggacatggccaggctacccagcccaaccaaggagaagcctcccccaccacccccaggtggaggcaaggacctgttctatgtgagccgtccacccttggctaggtcttccccagcttactgcacaagctcttctgacatcacagagccagagcagaagatgctatctgtgaacaagtcagtgtccatgctggacttgcagggggatgggccaggtggcaggttgaacagtagctctgtctctaaccttgctgctgtgggtgacctactccactccagccaggcctcactgaccgcagccttgggcctaaggcctgcaccagctggtaggctatctcaggggtcaggctccagcattacagctgcaggcatgaggttaagccagatgggggtcaccacagatggtgttccagcccagcagcttaggattcccctctccttccagaacccactgttccacatggctgctgacggtccagggccaccaggtggtcatgggggtggaggtggccacggtcccccttcctcacaccaccaccaccaccatcatcaccaccacaggggtggggagccaccaggggacacctttgccccattccatggctatagcaagtctgaggacctctcttcaggtgtgcccaagcccccagctgcttctatcctgcacagccacagctactctgatgagtttgggccctcagggacagacttcaccaggagacagctatccctgcaggacaatctgcagcacatgttgtctcccccacagatcacaattggcccacagaggccagctccctctggcccagggggtgggtcaggtggtggatctggaggcgggggtgggggccagccacccccacttcagaggggcaagtcccagcaactgacagtctcagctgcccagaagccccgaccatcctctggtaacctactacagtccccagagccatcctatggtccagctaggccaagacagcagagcctcagcaaggagggcagcattgggggtagtggtggctcgggtggcggaggtgggggtgggctcaagccctcaatcaccaagcagcactcccagacacccagcaccttgaaccccacaatgccagcttctgagaggacagtggcctgggtctccaacatgccacacctgtcggctgacattgagtcagcccacattgagagggaagagtacaagctcaaggagtactccaagtccatggatgagtctaggctggacagggtgaaagagtacgaagaggagatacacagcctgaaggagcggctgcacatgagcaacagaaagctggaggagtatgagcgtaggctcctgtcccaggaggagcagacatctaagatcctgatgcagtaccaggcaagactggagcagtcagagaagaggctaaggcagcagcaggctgagaaggactcccagatcaagagcatcattggcagactgatgctggtggaggaggagctgaggagggaccacccagctatggctgagcccctgccagagcccaagaagcgcctgctagatgctcagagggggtccttcccaccatgggtccagcagaccagggtgtaa

SEQ ID NO: 23 - protein, 546 aa, synthetic construct
LNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQRGSFPPWVQQTRV

SEQ ID NO: 24 - protein, 581 aa, synthetic construct
LNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQERQLPPLGPTNPRVTLAPPWNGLAPPAPPPPPRLQITENGEFRNTADH

SEQ ID NO: 25 - protein, 523 aa, synthetic construct
LNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGSPSLQADAGGGGAAPGPPRHG

SEQ ID NO: 26 - protein, 537 aa, synthetic construct
LNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQLLIR

(19) United States
(12) Patent Application Publication
(10) Pub. No.: US 2025/0230467 A1
(43) Pub. Date: Jul. 17, 2025

(54) METHODS AND MATERIALS FOR TREATING SYNGAP1-ASSOCIATED NEURODEVELOPMENTAL DISORDERS

(71) Applicant: The Johns Hopkins University, Baltimore, MD (US)

(72) Inventors: Ingie Hong, North Bethesda, MD (US); Richard Huganir, Cockeysville, MD (US); Yoichi Araki, Cockeysville, MD (US); Richard C. Johnson, Baltimore, MD (US)

(21) Appl. No.: 18/854,168

(22) PCT Filed: Apr. 5, 2023
(86) PCT No.: PCT/US2023/065364

§ 371 Date: Oct. 4, 2024

Related U.S. Application Data
(60) Provisional application No. 63/327,624, filed on Apr. 5, 2022.

(51) Int. Cl. C12N 15/86; A61K 48/00; A61P 25/00; C07K 14/47

(52) U.S. Cl. CPC C12N 15/86; A61K 48/0058; A61P 25/00; C07K 14/4702; C12N 2750/14143; C12N 2830/008

(57) ABSTRACT

This document provides methods and materials for treating SYNGAP1-associated neurodevelopmental disorders (NDDs; e.g., SYNGAP1-related intellectual disability (SRID)). For example, viral vectors (e.g., adeno-associated viral (AAV) vectors) that include (e.g., are designed to include) nucleic acid encoding a truncated Syngap1 polypeptide (e.g., Syngap1-B:α1 polypeptide) are provided. In some cases, one or more viral vectors provided herein (e.g., AAV vectors that include nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) to treat the mammal.

### CROSS-REFERENCE TO RELATED APPLICATIONS

This application claims the benefit of U.S. Patent Application Ser. No. 63/327,624, filed on Apr. 5, 2022. The disclosure of the prior application is considered part of, and is incorporated by reference in, the disclosure of this application.

### STATEMENT REGARDING FEDERAL FUNDING

This invention was made with government support under MH112151 awarded by the National Institutes of Health. The government has certain rights in the invention.

### SEQUENCE LISTING

This application contains a Sequence Listing that has been submitted electronically as an XML file named “44807-0418WO1.XML.” The XML file, created on Apr. 2, 2023, is 60000 bytes in size. The material in the XML file is hereby incorporated by reference in its entirety.

### TECHNICAL FIELD

This document relates to methods and materials for treating SYNGAP1-associated neurodevelopmental disorders (NDDs; e.g., SYNGAP1-related intellectual disability (SRID)).

For example, this document provides viral vectors (e.g., adeno-associated viral (AAV) vectors) that include (e.g., are designed to include) nucleic acid encoding a truncated Syngap1 polypeptide (e.g., a Syngap1-B:α1 polypeptide). In some cases, one or more viral vectors provided herein (e.g., AAV vectors that include nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) to treat the mammal.

### BACKGROUND INFORMATION

SYNGAP1-related Intellectual Disability (SRID, MRD5) is a severe neurodevelopmental disorder (NDD) characterized by intellectual disability (ID), autism spectrum disorder (ASD), and epilepsy (Vlaskamp et al., Neurology, 92(2):e96-e107 (2019); and Jimenez-Gomez et al., J. Neurodev. Disord., 11(1):18 (2019)). SRID is estimated to account for 0.5-1% of all NDD and ˜1% of the −200 million ID cases worldwide (UK-DDD-study, Nature, 519(7542):223-8 (2015); Carvill et al., Nat. Genet., 45(7):825-30 (2013); Berryer et al., Hum. Mutat., 34(2):385-94 (2013); Hamdan et al., Am. J. Hum. Genet., 88(3):306-16 (2011); Hamdan et al., N. Engl. J. Med., 360(6):599-605 (2009); and Lopez-Rivera et al., Brain, 143(4):1099-105 (2020)) with no disease modifying treatment available.

### SUMMARY

Advances in human genetics have revealed high penetrance loss-of-function (LoF) mutations in SYNGAP1 lead to severe intellectual disability (ID)/autism spectrum disorder (ASD)/epilepsy at extremely high penetrance. High-penetrance monogenic disorder are typically good candidates for gene therapy. However, gene therapy for SYNGAP1-associated NDDs faces many challenges including brain penetration of the gene therapy components and the large size of the full-length SYNGAP1 transcript (4,029 base pairs (bp)) and of the Syngap1 polypeptide (1,343 amino acids (aa)). This disclosure is based, at least in part, on the discoveries that nucleic acid encoding a truncated Syngap1 polypeptide (e.g., a Syngap1-B:α1 polypeptide which is an N-terminal truncated version of a Syngap1 polypeptide that arises from an alternative TSS site and c-terminal alternative splice form of a Syngap1-α1 isoform) can be packaged in an AAV vector, and that an AAV vector including nucleic acid encoding a truncated Syngap1 polypeptide (e.g., a Syngap1-B:α1 polypeptide) can be used to treat SYNGAP1-associated NDDs.

This document relates to methods and materials involved in treating a mammal (e.g., a human) having, or at risk for developing, a SYNGAP1-associated NDD (e.g., SRID). For example, this document provides viral vectors (e.g., AAV vectors) that include (e.g., are designed to include) nucleic acid encoding a truncated Syngap1 polypeptide (e.g., a Syngap1-B:α1 polypeptide). This document also provides methods for making and using such viral vectors (e.g., AAV vectors that include nucleic acid encoding a truncated Syngap1 polypeptide such as a Syngap1-B:α1 polypeptide). In some cases, one or more viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD such as SRID (e.g., to treat the mammal). For example, one or more viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) to increase the level of one or more Syngap1 polypeptides (e.g., one or more truncated Syngap1 polypeptides such as Syngap1-B:α1 polypeptides) within neurons within the mammal.

As demonstrated herein, AAV vectors can be used to robustly express Syngap1 polypeptides in neurons. For example, AAV vectors (e.g., self-complementary AAV (scAAV) vectors can be used to robustly express a truncated Syngap1 polypeptide in neurons to rescue SYNGAP1-disease model phenotypes in cultured neurons. Having the ability to increase the level of one or more Syngap1 polypeptides (e.g., one or more truncated Syngap1 polypeptides such as Syngap1-B:α1 polypeptides) within neurons within the mammal provides a unique and unrealized opportunity to treat SYNGAP1-associated NDDs (e.g., SRID).

In general, one aspect of this document features viral vectors comprising a nucleic acid encoding a truncated Syngap1 polypeptide. The viral vector can be an AAV vector, a retroviral vector, a rhabdovirus-based vector, an adenovirus vector, or a herpes simplex virus vector. The viral vector can be an AAV vector. The truncated Syngap1 polypeptide can be a Syngap1-B:α1 polypeptide, a Syngap1-B:α2 polypeptide, a Syngap1-B:α3 polypeptide, a Syngap1-B:-β. polypeptide, a Syngap1-B:7 polypeptide, a Syngap1-α1 polypeptide, a Syngap1-α2 polypeptide, a Syngap1-α3 polypeptide, a Syngap1-β polypeptide, a Syngap1-y polypeptide, or a Syngap1-exon14-20 polypeptide. The truncated Syngap1 polypeptide can comprise, consist essentially of, or consist of an amino acid sequence set forth in any one of SEQ ID NOs:7-11 or SEQ ID NOs:23-26. The nucleic acid encoding the truncated Syngap1 polypeptide can comprise, consist essentially of, or consist of a nucleic acid sequence set forth in any one of SEQ ID NOs:1-6 or SEQ ID NO:22. The nucleic acid encoding the truncated Syngap1 polypeptide can be operably linked to a promoter. The promoter can be a neuron-specific promoter. The promoter can be a CaMKIIα promoter, a SYN1 promoter, or a SYNGAP1 promoter. The viral vector can include an optimized inverted terminal repeat (ITR). The viral vector can include an optimized 3′ untranslated region (UTR). The optimized 3′ UTR can be a SV403′ UTR, a hGH3′ UTR, a BGH3′ UTR, a rbGlob 3′ UTR, a CW3SL3′ UTR, or a 2×SNRP 3′ UTR. The viral vector can include a nucleic acid sequence that can targeted by an anti-sense oligonucleotide (ASO). The nucleic acid sequence that can be targeted by the ASO can be a synthetic nucleic acid sequence. The nucleic acid sequence that can be targeted by the ASO can be sequence that is not an endogenous human sequence. The ASO can include a sequence set forth in any one of SEQ ID NOs:12-21.

In another aspect, this document features methods for treating a mammal having or at risk of developing a SYNGAP1-associated NDD. The methods can include, or consist essentially of, administering to said mammal a viral vector comprising a nucleic acid encoding a truncated Syngap1 polypeptide. The mammal can be a human. The human can be an infant (e.g., a newborn). The SYNGAP1-associated NDD can be a SYNGAP1-related intellectual disability (SRID), SYNGAP1-related autism spectrum disorder (ASD), SYNGAP1-related epilepsy, a sleep disorder, or schizophrenia. The administering can include an intracerebroventricular (ICV) injection, an intravenous (IV) injection, or an intracranial (IC) injection. The viral vector can be an AAV vector, a retroviral vector, a rhabdovirus-based vector, an adenovirus vector, or a herpes simplex virus vector. The viral vector can be an AAV vector. The truncated Syngap1 polypeptide can be a Syngap1-B:α1 polypeptide, a Syngap1-B:α2 polypeptide, a Syngap1-B:α3 polypeptide, a Syngap1-B:β polypeptide, a Syngap1-B:γ polypeptide, a Syngap1-α1 polypeptide, a Syngap1-α2 polypeptide, a Syngap1-α3 polypeptide, a Syngap1-β polypeptide, a Syngap1-7 polypeptide, or a Syngap1-exon14-20 polypeptide. The truncated Syngap1 polypeptide can comprise, consist essentially of, or consist of an amino acid sequence set forth in any one of SEQ ID NOs:7-11 or SEQ ID NOs:23-26. The nucleic acid encoding the truncated Syngap1 polypeptide can comprise, consist essentially of, or consist of a nucleic acid sequence set forth in any one of SEQ ID NOs:1-6 or SEQ ID NO:22. The nucleic acid encoding the truncated Syngap1 polypeptide can be operably linked to a promoter. The promoter can be a neuron-specific promoter. The promoter can be a CaMIKIIα promoter, a SYN1 promoter, or a SYNGAP1 promoter. The viral vector can include an optimized ITR. The viral vector can include an optimized 3′ UTR. The optimized 3′ UTR can be a SV403′ UTR, a hGH3′ UTR, a BGH3′ UTR, a rbGlob 3′ UTR, a CW3SL3′ UTR, or a 2×SNRP 3′ UTR. The viral vector can include a nucleic acid sequence that can targeted by an ASO. The nucleic acid sequence that can be targeted by the ASO can be a synthetic nucleic acid sequence. The nucleic acid sequence that can be targeted by the ASO can be sequence that is not an endogenous human sequence. The ASO can include a sequence set forth in any one of SEQ ID NOs:12-21.

In another aspect, this document features methods for increasing a level of a truncated Syngap1 polypeptide within neurons of a mammal. The methods can include, or consist essentially of, administering to a mammal a viral vector comprising a nucleic acid encoding a truncated Syngap1 polypeptide. The mammal can be a human. The human can be an infant (e.g., a newborn). The increase in the level of the truncated Syngap1 polypeptide can be from about 1.2 fold to about 20 fold relative to a level of the truncated Syngap1 polypeptide in the mammal prior to being administered the viral vector. The mammal can have a SYNGAP1-associated NDD selected from the group consisting of a SRID, SYNGAP1-related ASD, SYNGAP1-related epilepsy, a sleep disorder, and schizophrenia. The administering can include an ICV injection, an IV injection, or an IC injection. The viral vector can be an AAV vector, a retroviral vector, a rhabdovirus-based vector, an adenovirus vector, or a herpes simplex virus vector. The viral vector can be an AAV vector. The truncated Syngap1 polypeptide can be a Syngap1-B:α1 polypeptide, a Syngap1-B:α2 polypeptide, a Syngap1-B:α3 polypeptide, a Syngap1-B:β polypeptide, a Syngap1-B:γ polypeptide, a Syngap1-α1 polypeptide, a Syngap1-α2 polypeptide, a Syngap1-α3 polypeptide, a Syngap1-β polypeptide, a Syngap1-y polypeptide, or a Syngap1-exon14-20 polypeptide. The truncated Syngap1 polypeptide can comprise, consist essentially of, or consist of an amino acid sequence set forth in any one of SEQ ID NOs:7-11 or SEQ ID NOs:23-26. The nucleic acid encoding the truncated Syngap1 polypeptide can comprise, consist essentially of, or consist of a nucleic acid sequence set forth in any one of SEQ ID NOs:1-6 or SEQ ID NO:22. The nucleic acid encoding the truncated Syngap1 polypeptide can be operably linked to a promoter. The promoter can be a neuron-specific promoter. The promoter can be a CaMKIIα promoter, a SYN1 promoter, or a SYNGAP1 promoter. The viral vector can include an optimized ITR. The viral vector can include an optimized 3′ UTR. The optimized 3′ UTR can be a SV403′ UTR, a hGH3′ UTR, a BGH3′ UTR, a rbGlob 3′ UTR, a CW3SL3′ UTR, or a 2×SNRP 3′ UTR. The viral vector can include a nucleic acid sequence that can targeted by an ASO. The nucleic acid sequence that can be targeted by the ASO can be a synthetic nucleic acid sequence. The nucleic acid sequence that can be targeted by the ASO can be sequence that is not an endogenous human sequence. The ASO can include a sequence set forth in any one of SEQ ID NOs:12-21.

In another aspect, this document features uses of a composition comprising a viral vector comprising a nucleic acid encoding a truncated Syngap1 polypeptide to treat a mammal having a SYNGAP1-associated NDD. The mammal can be a human. The human can be an infant (e.g., a newborn). The SYNGAP1-associated NDD can be a SRID, SYNGAP1-related ASD, SYNGAP1-related epilepsy, a sleep disorder, or schizophrenia. The viral vector can be an AAV vector, a retroviral vector, a rhabdovirus-based vector, an adenovirus vector, or a herpes simplex virus vector. The viral vector can be an AAV vector. The truncated Syngap1 polypeptide can be a Syngap1-B:α1 polypeptide, a Syngap1-B:α2 polypeptide, a Syngap1-B:α3 polypeptide, a Syngap1-B:β polypeptide, a Syngap1-B:γ polypeptide, a Syngap1-α1 polypeptide, a Syngap1-α2 polypeptide, a Syngap1-α3 polypeptide, a Syngap1-β polypeptide, a Syngap1-7 polypeptide, or a Syngap1-exon14-20 polypeptide. The truncated Syngap1 polypeptide can comprise, consist essentially of, or consist of an amino acid sequence set forth in any one of SEQ ID NOs:7-11 or SEQ ID NOs:23-26. The nucleic acid encoding the truncated Syngap1 polypeptide can comprise, consist essentially of, or consist of a nucleic acid sequence set forth in any one of SEQ ID NOs:1-6 or SEQ ID NO:22. The nucleic acid encoding the truncated Syngap1 polypeptide can be operably linked to a promoter. The promoter can be a neuron-specific promoter. The promoter can be a CaMKIIα promoter, a SYN1 promoter, or a SYNGAP1 promoter. The viral vector can include an optimized ITR. The viral vector can include an optimized 3′ UTR. The optimized 3′ UTR can be a SV403′ UTR, a hGH3′ UTR, a BGH3′ UTR, a rbGlob 3′ UTR, a CW3SL3′ UTR, or a 2×SNRP 3′ UTR. The viral vector can include a nucleic acid sequence that can targeted by an ASO. The nucleic acid sequence that can be targeted by the ASO can be a synthetic nucleic acid sequence. The nucleic acid sequence that can be targeted by the ASO can be sequence that is not an endogenous human sequence. The ASO can include a sequence set forth in any one of SEQ ID NOs:12-21.

In another aspect, this document features viral vectors comprising a nucleic acid encoding a truncated Syngap1 polypeptide for use in the preparation of a medicament for treating a mammal having a SYNGAP1-associated NDD. The mammal can be a human. The human can be an infant (e.g., a newborn). The SYNGAP1-associated NDD can be a SRID, SYNGAP1-related ASD, SYNGAP1-related epilepsy, a sleep disorder, or schizophrenia. The viral vector can be an AAV vector, a retroviral vector, a rhabdovirus-based vector, an adenovirus vector, or a herpes simplex virus vector. The viral vector can be an AAV vector. The truncated Syngap1 polypeptide can be a Syngap1-B:α1 polypeptide, a Syngap1-B:α2 polypeptide, a Syngap1-B:α3 polypeptide, a Syngap1-B:β polypeptide, a Syngap1-B:γ polypeptide, a Syngap1-α1 polypeptide, a Syngap1-α2 polypeptide, a Syngap1-α3 polypeptide, a Syngap1-β polypeptide, a Syngap1-γ polypeptide, or a Syngap1-exon14-20 polypeptide. The truncated Syngap1 polypeptide can comprise, consist essentially of, or consist of an amino acid sequence set forth in any one of SEQ ID NOs:7-11 or SEQ ID NOs:23-26. The nucleic acid encoding the truncated Syngap1 polypeptide can comprise, consist essentially of, or consist of a nucleic acid sequence set forth in any one of SEQ ID NOs:1-6 or SEQ ID NO:22. The nucleic acid encoding the truncated Syngap1 polypeptide can be operably linked to a promoter. The promoter can be a neuron-specific promoter. The promoter can be a CaMKIIα promoter, a SYN1 promoter, or a SYNGAP1 promoter. The viral vector can include an optimized ITR. The viral vector can include an optimized 3′ UTR. The optimized 3′ UTR can be a SV403′ UTR, a hGH3′ UTR, a BGH3′ UTR, a rbGlob 3′ UTR, a CW3SL3′ UTR, or a 2×SNRP 3′ UTR. The viral vector can include a nucleic acid sequence that can targeted by an ASO. The nucleic acid sequence that can be targeted by the ASO can be a synthetic nucleic acid sequence. The nucleic acid sequence that can be targeted by the ASO can be sequence that is not an endogenous human sequence. The ASO can include a sequence set forth in any one of SEQ ID NOs:12-21.

In another aspect, this document features viral vectors comprising a nucleic acid encoding a truncated Syngap1 polypeptide for use in the treatment of a mammal having a SYNGAP1-associated NDD. The mammal can be a human. The human can be an infant (e.g., a newborn). The SYNGAP1-associated NDD can be a SRID, SYNGAP1-related ASD, SYNGAP1-related epilepsy, a sleep disorder, or schizophrenia. The viral vector can be an AAV vector, a retroviral vector, a rhabdovirus-based vector, an adenovirus vector, or a herpes simplex virus vector. The viral vector can be an AAV vector. The truncated Syngap1 polypeptide can be a Syngap1-B:α1 polypeptide, a Syngap1-B:α2 polypeptide, a Syngap1-B:α3 polypeptide, a Syngap1-B:β polypeptide, a Syngap1-B:γ polypeptide, a Syngap1-α1 polypeptide, a Syngap1-α2 polypeptide, a Syngap1-α3 polypeptide, a Syngap1-β polypeptide, a Syngap1-γ polypeptide, or a Syngap1-exon14-20 polypeptide. The truncated Syngap1 polypeptide can comprise, consist essentially of, or consist of an amino acid sequence set forth in any one of SEQ ID NOs:7-11 or SEQ ID NOs:23-26. The nucleic acid encoding the truncated Syngap1 polypeptide can comprise, consist essentially of, or consist of a nucleic acid sequence set forth in any one of SEQ ID NOs:1-6 or SEQ ID NO:22. The nucleic acid encoding the truncated Syngap1 polypeptide can be operably linked to a promoter. The promoter can be a neuron-specific promoter. The promoter can be a CaMKIIα promoter, a SYN1 promoter, or a SYNGAP1 promoter. The viral vector can include an optimized ITR. The viral vector can include an optimized 3′ UTR. The optimized 3′ UTR can be a SV403′ UTR, a hGH3′ UTR, a BGH3′ UTR, a rbGlob 3′ UTR, a CW3SL3′ UTR, or a 2×SNRP 3′ UTR. The viral vector can include a nucleic acid sequence that can targeted by an ASO. The nucleic acid sequence that can be targeted by the ASO can be a synthetic nucleic acid sequence. The nucleic acid sequence that can be targeted by the ASO can be sequence that is not an endogenous human sequence. The ASO can include a sequence set forth in any one of SEQ ID NOs:12-21.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. AAV-Syngap1 screen. Several different strategies were screened for robust expression of exogenous Flag-Syngap1 in neurons, including 3′ UTR elements (CW3SL/2×SNRP), ITR modifications (ITR), and split AAV reconstitution. Although all candidates expressed Flag-Syngap1 with the correct synaptic localization and colocalized with native Syngap1, the AAV-CaMKIIα-Syngap1-CW3SL showed robust and broad expression. AAV-PSD95 intrabody was used as a positive control. Scale bar: 3 μm.

FIGS. 2A-2B. AAV-Syngap1 expression in primary cultured neurons. FIG. 2A: Primary cultured neurons were treated with AAV-Syngap1 variants, lysed and immunoblotted to with anti-FLAG antibody (detect exogenous expressed Syngap1) and anti-Syngap1 antibody (detect both exogenous and endogenous Syngap1). FIG. 2B: Quantification of expressed Syngap1 polypeptide. The strongest expression was from AAV-CaMKII-Flag-SG-Bal-CW3SL at 700% of endogenous levels.

FIGS. 3A-3B. ‘Kill-switch’ ASO diminished AAV-SYNGAP1 expression in a dose-dependent-manner. FIG. 3A: Cultured neurons were treated by AAV-SYNGAP1 at DIV13. After 2 days, neurons were further treated by an ASO (1×: 30 M or 3×: 90M) targeted at a unique sequence in ASO-SYNGAP1. Neurons were lysed and subjected to immunoblotting with anti-FLAG antibody (to detect exogenously expressed SYNGAP1) and anti-SYNGAP1 antibody (to detect both exogenous and endogenous SYNGAP1). FIG. 3B.

Quantification of expressed SYNGAP1 polypeptide. FLAG and SYNGAP1 blots both showed an ASO dose-dependent suppression of FLAG-SYNGAP1 expression. Suppression reached 7-20 fold of AAV-only treated exogenous SYNGAP1 polypeptide expression.

FIG. 4. AAV-SYNGAP1 for each c-terminal isoform robustly expressed SYNGAP1 protein. Rat hippocampal neurons were infected with AAV-SYNGAP1 V2 coding the four major c-terminal isoforms, α1, α2, β, and γ. Protein expression was tested with Western blots using Flag, Syngap1-α1, Syngap1-α2, Syngap1-β, Syngap1-γ, total Syngap1, and GAPDH antibodies.

FIGS. 5A-5B. Improved SynGAP expression in AAV-SYNGAP1 with alternative capsids. FIG. 5A: Rat hippocampal neurons were infected by AAV-SYNGAP1 with AAV2/9, DJ, and B10 capsids. FIG. 5B: Quantification ofFLAG-SynGAP expression levels normalized to AAV2/9-SynGAP.

FIGS. 6A-6C. FIG. 6A: SYNGAP1 full-length and exon 14-20 minigene DIS—CC-PBM (disordered region-coiled coil domain-PDZ binding module). FIG. 6B: Spine size was enlarged upon LTP (WT). Note that SYNGAP1 KD led to enlarged basal spine sizes which occluded LTP-induced spine enlargement. Both the full-length and exon 14-20 minigene SYNGAP1 almost equally rescued this deficit. FIG. 6C: Minigene SynGAP dynamics during LTP, showing similar dispersal from spines with full-length.

FIG. 7. scAAV-SYNGAP1(exon 14-20 minigene) robustly expressed truncated SYNGAP1 protein. Rat hippocampal neurons were infected with AAV-SYNGAP1, AAVJH-SYNGAP V3, AAVJH2-SYNGAP1 V3 and two scAAV-SYNGAP1s (exon 14-20 minigene). Protein expression was tested with Western blots using Flag, Syngap1, Syngap1-α1, and GAPDH antibodies.

### DETAILED DESCRIPTION

This document provides methods and materials involved in treating a mammal (e.g., a human) having, or at risk for developing, a SYNGAP1-associated NDD (e.g., SRID). For example, this document provides viral vectors (e.g., AAV vectors) that include (e.g., are 25 designed to include) nucleic acid encoding a truncated Syngap1 polypeptide (e.g., a Syngap1-B:α1 polypeptide). This document also provides methods for making and using such viral vectors (e.g., AAV vectors that include nucleic acid encoding a truncated Syngap1 polypeptide such as a Syngap1-B:α1 polypeptide). In some cases, one or more viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD such as SRID (e.g., to treat the mammal). For example, one or more viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) to increase the level of one or more Syngap1 polypeptides (e.g., one or more truncated Syngap1 polypeptides such as Syngap1-B:α1 polypeptides) within neurons within the mammal.

In some cases, a viral vector (e.g., AAV vector) provided herein (e.g., a viral vector that includes nucleic acid encoding a truncated Syngap1 polypeptide such as a Syngap1-B:α1 polypeptide) can be neurotropic (e.g., to a mammal being treated as described herein). For example, a viral vector provided herein can infect (e.g., and express a truncated Syngap1 polypeptide such as a Syngap1-B:α1 polypeptide) within a neuron such as an excitatory neuron.

In some cases, a viral vector (e.g., AAV vector) provided herein (e.g., a viral vector that includes nucleic acid encoding a truncated Syngap1 polypeptide such as a Syngap1-B:α1 polypeptide) can be replication defective.

In some cases, a viral vector (e.g., AAV vector) provided herein (e.g., a viral vector that includes nucleic acid encoding a truncated Syngap1 polypeptide such as a Syngap1-B:α1 polypeptide) can infect non-dividing cells (e.g., can infect only non-dividing cells).

In some cases, a viral vector (e.g., AAV vector) provided herein (e.g., a viral vector that includes nucleic acid encoding a truncated Syngap1 polypeptide such as a Syngap1-B:α1 polypeptide) can be non-pathogenic (e.g., to a mammal being treated as described herein).

A viral vector provided herein (e.g., a viral vector that includes nucleic acid encoding a truncated Syngap1 polypeptide such as a Syngap1-B:α1 polypeptide) can be derived from (e.g., can include genomic elements such as nucleic acids encoding a polypeptide (or fragments thereof)) from any appropriate viral vector. Examples of viral vectors that can include (e.g., can be designed to include) nucleic acid encoding a truncated Syngap1 polypeptide (e.g., a Syngap1-B:α1 polypeptide) include, without limitation, AAV vectors, retroviral vectors, rhabdovirus-based vectors, adenovirus vectors, and herpes simplex virus vectors. When a viral vector provided herein is an AAV vector, the AAV vector can be any AAV serotype. In some cases, an AAV vector can have a synthetic (e.g., a hybrid) serotype. Examples of AAV vector serotypes include, without limitation, AAV2, AAV2/9, AAV9, AAV1, AAVrh10, AAV.B10, and AAV-DJ. When a viral vector provided herein is an AAV vector, the AAV vector can be a self-complementary AAV (scAAV) vector.

A viral vector provided herein (e.g., a viral vector that includes nucleic acid encoding a truncated Syngap1 polypeptide such as a Syngap1-B:α1 polypeptide) can include nucleic acid encoding any appropriate truncated Syngap1 polypeptide. In some cases, a nucleic acid encoding a truncated Syngap1 polypeptide (e.g., a Syngap1-B:α1 polypeptide) can be less than 4000 bp in length. For example, a nucleic acid encoding a truncated Syngap1 polypeptide (e.g., a Syngap1-B:α1 polypeptide) can be from about 1500 bp to about 4000 bp in length (e.g., from about 1500 bp to about 3500 bp, from about 1500 bp to about 3200 bp, from about 1500 bp to about 3000 bp, from about 1500 bp to about 2700 bp, from about 1500 bp to about 2500 bp, from about 1500 bp to about 2200 bp, from about 1500 bp to about 2000 bp, from about 1500 bp to about 1800 bp, from about 1800 bp to about 4000 bp, from about 2000 bp to about 4000 bp, from about 2300 bp to about 4000 bp, from about 2500 bp to about 4000 bp, from about 2800 bp to about 4000 bp, from about 3000 bp to about 4000 bp, from about 3200 bp to about 4000 bp, from about 3500 bp to about 4000 bp, from about 1800 bp to about 3500 bp, from about 2000 bp to about 3200 bp, from about 2300 bp to about 3000 bp, from about 1800 bp to about 2000 bp, from about 2000 bp to about 2500 bp, from about 2500 bp to about 3000 bp, or from about 3000 bp to about 3500 bp in length).

In some cases, a nucleic acid encoding a truncated Syngap1 polypeptide (e.g., a Syngap1-B:α1 polypeptide) can encode a truncated Syngap1 polypeptide that is less than 1300 aa in length. For example, a truncated Syngap1 polypeptide (e.g., a Syngap1-B:α1 polypeptide) can be from about 500 aa to about 1300 aa in length (e.g., from about 500 aa to about 1000 aa, from about 500 aa to about 800 aa, from about 700 aa to about 1300 aa, from about 1000 aa to about 1300 aa, from about 700 aa to about 1200 aa, from about 800 aa to about 1000 aa, from about 600 aa to about 800 aa, from about 800 aa to about 1000 aa, or from about 800 aa to about 1200 aa in length).

A truncated Syngap1 polypeptide that can be encoded by a viral vector provided herein (e.g., a viral vector that includes nucleic acid encoding a truncated Syngap1 polypeptide such as a Syngap1-B:α1 polypeptide) can be any appropriate truncated Syngap1 polypeptide. In some cases, a truncated Syngap1 polypeptide can be a naturally occurring Syngap1 polypeptide isoform. In some cases, a truncated Syngap1 polypeptide can be derived from any Syngap1 polypeptide isoform (e.g., Syngap1-A, Syngap1-B, and Syngap1-C isoforms). For example, a truncated Syngap1 polypeptide can have an N-terminal truncation. For example, a nucleic acid encoding a truncated Syngap1 polypeptide can lack one or more of exons 8-14 (e.g., exons 8-14 of a SYNGAP1 gene). In some cases, a truncated Syngap1 polypeptide can include an intrinsically disordered region, coiled-coil, PDZ-binding module (DIS—CC-PBM) domain. Examples of truncated Syngap1 polypeptides that can be encoded by a viral vector (e.g., an AAV vector) provided herein include, without limitation, Syngap1-B:α1 polypeptides, Syngap1-B:α2 polypeptides, Syngap1-B:α3 polypeptides, Syngap1-B:β polypeptides, Syngap1-B:7 polypeptides, Syngap1-α1 polypeptides, Syngap1-α2 polypeptides, Syngap1-α3 polypeptides, Syngap1-β polypeptides, Syngap1-γ polypeptides, Syngap1-exon14-20 polypeptides (e.g., Syngap1-exon14-20al polypeptides, Syngap1-exon14-20:α2 polypeptides, Syngap1-exon14-20:α3 polypeptides, Syngap1-exon14-20:p polypeptides, and Syngap1-exon14-20:7 polypeptides). Representative nucleic acids that can encode a truncated Syngap1 polypeptide and can be included in a viral vector (e.g., an AAV vector) provided herein are set forth in SEQ ID NOs:1-6 or SEQ ID NO:22 (see, e.g., Example 2). Representative truncated Syngap1 polypeptides that can be encoded by a viral vector (e.g., an AAV vector) provided herein are set forth in SEQ ID NOs:7-11 or SEQ ID NOs:23-26 (see, e.g., Example 2).

A truncated Syngap1 polypeptide (e.g., a Syngap1-B:α1 polypeptide) that can be encoded by a viral vector provided herein (e.g., a viral vector that includes nucleic acid encoding a truncated Syngap1 polypeptide such as a Syngap1-B:α1 polypeptide), can have any appropriate amino acid sequence. For example, a truncated Syngap1 polypeptide (e.g., a Syngap1-B:α1 polypeptide) that can be encoded by a viral vector (e.g., an AAV vector) provided herein can have an amino acid sequence as set forth in any one of SEQ ID NOs:7-11 or SEQ ID NOs:23-26 (see, e.g., Example 2). In some cases, a truncated Syngap1 polypeptide (e.g., a Syngap1-B:α1 polypeptide) can have a sequence that deviates from a sequence set forth in any one of SEQ ID NOs:7-11 or SEQ ID NOs:23-26 (e.g., can comprise or consist essentially of an amino acid sequence set forth in any one of SEQ ID NOs:7-11 or SEQ ID NOs:23-26). For example, an amino acid sequence of a truncated Syngap1 polypeptide (e.g., a Syngap1-B:α1 polypeptide) can have at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, or about 99% sequence identity) to the amino acid sequence set forth in any one of SEQ ID NOs:7-11 or SEQ ID NOs:23-26, provided that the truncated Syngap1 polypeptide retains the ability to localize to synapses and support synaptic plasticity. Percent sequence identity is calculated by determining the number of matched positions in aligned amino acid sequences, dividing the number of matched positions by the total number of aligned amino acids, and multiplying by 100. A matched position refers to a position in which identical amino acid residues occur at the same position in aligned sequences. Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web.

BLAST searches or alignments can be performed to determine percent sequence identity between an amino acid and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between an amino acid sequence and another sequence, the default parameters of the respective programs can be used.

In some cases, a truncated Syngap1 polypeptide (e.g., a Syngap1-B:α1 polypeptide) that can be encoded by a viral vector provided herein (e.g., a viral vector that includes nucleic acid encoding a truncated Syngap1 polypeptide such as a Syngap1-B:α1 polypeptide) can have an amino acid sequence that includes one or more modifications (e.g., deletions, insertions, and substitutions) to the amino acid sequence set forth in any one of SEQ ID NOs:7-11 or SEQ ID NOs:23-26. For example, a truncated Syngap1 polypeptide (e.g., a Syngap1-B:α1 polypeptide) can consist of the amino acid sequence set forth in any one of SEQ ID NOs:7-11 or SEQ ID NOs:23-26, except that the truncated Syngap1 polypeptide includes one, two, three, four, five, or more amino acid substitutions within the articulated sequence of the sequence identifier (e.g., any one of SEQ ID NOs:7-11 or SEQ ID NOs:23-26), has one, two, three, four, five, or more amino acid residues preceding the articulated sequence of the sequence identifier (e.g., any one of SEQ ID NOs:7-11 or SEQ ID NOs:23-26), and/or has one, two, three, four, five, or more amino acid residues following the articulated sequence of the sequence identifier (e.g., any one of SEQ TD NOs:7-11 or SEQ ID NOs:23-26), provided that the truncated Syngap1 polypeptide retains the ability to localize to synapses and support synaptic plasticity.

A viral vector provided herein (e.g., a viral vector that includes nucleic acid encoding a truncated Syngap1 polypeptide such as a Syngap1-B:α1 polypeptide) can include one or more regulatory elements operably linked to nucleic acid encoding a truncated Syngap 1 polypeptide (e.g., a Syngap1-B:α1 polypeptide). Such regulatory elements can include promoter sequences, enhancer sequences, untranslated regions (UTRs; e.g., a modified 5′ UTR and/or a modified 3′ UTR), inverted terminal repeats (ITRs; e.g., modified ITRs), response elements, signal peptides, internal ribosome entry sequences, polyadenylation signals, terminators, and inducible elements that modulate expression (e.g., transcription or translation) of a nucleic acid. In some cases, a regulatory element can be a woodchuck hepatitis virus post-transcriptional regulatory element (WPRE) such as a WPREmut6ΔΔTG.

In some cases, a regulatory element can be as described elsewhere (see, e.g., Hamilton et al., Front. Immunol., 12:675897 (2021); Zanta-Boussif et al., Gene Ther., 16(5):605-19 (2009); Richter et al., Cell, 163(2):292-300 (2015); and Wu et al., eLife, 8:e45396 (2019)). The choice of regulatory element(s) that can be included in a viral vector genome depends on several factors, including, without limitation, inducibility, targeting, and the level of expression desired.

In some cases, a viral vector provided herein (e.g., a viral vector that includes nucleic acid encoding a truncated Syngap1 polypeptide such as a Syngap1-B:α1 polypeptide) can include a promoter. For example, a promoter can be included in a viral vector provided herein to facilitate transcription of nucleic acid encoding a truncated Syngap1 polypeptide (e.g., a Syngap1-B:α1 polypeptide). A promoter can be a naturally occurring promoter or a recombinant promoter. A promoter can be a constitutive promoter or an inducible promoter. A promoter can be a ubiquitous promoter or a tissue/cell-specific promoter (e.g., a neuron-specific promoter). A promoter can be a full length promoter or a shortened promoter.

Examples of promoters that can increase expression of a polypeptide (e.g., a Syngap1-B:α1 polypeptide) from a nucleic acid sequence within a viral vector provided herein include, without limitation, CaMKIIα promoters, SYN1 promoters (e.g., human SYN1 promoters), and SYNGAP1 promoters. As used herein, “operably linked” refers to positioning of a regulatory element in a viral vector relative to a nucleic acid encoding a truncated Syngap1 polypeptide (e.g., a Syngap1-B:α1 polypeptide) in such a way as to permit or facilitate expression of the truncated Syngap1 polypeptide.

In some cases, a viral vector provided herein (e.g., a viral vector that includes nucleic acid encoding a truncated Syngap1 polypeptide such as a Syngap1-B:α1 polypeptide) can include an optimized ITR. Examples of optimized ITRs that can be included in a viral vector provided herein include, without limitation, ITR-S, ITRABC, and CpG-free ITR. In some cases, an optimized ITR that can be included in a viral vector that includes nucleic acid encoding a truncated Syngap1 polypeptide (e.g., a Syngap1-B:α1 polypeptide) can be as described elsewhere (see, e.g., Zhou et al., J. Am. Stat. Assoc., 112(517):169-187 (2017); and Pan et al., Gene Ther., doi: 10.1038/s41434-021-00296-0 (2021)).

In some cases, a viral vector provided herein (e.g., a viral vector that includes nucleic acid encoding a truncated Syngap1 polypeptide such as a Syngap1-B:α1 polypeptide) can include an optimized UTR (e.g., an optimized 3′ UTR). Examples of optimized UTRs that can be included in a viral vector provided herein include, without limitation, SV40, hGH, BGH, rbGlob, CW3SL, and 2×SNRP.

In some cases, a viral vector provided herein (e.g., a viral vector that includes nucleic acid encoding a truncated Syngap1 polypeptide such as a Syngap1-B:α1 polypeptide) also can include one or more additional nucleic acid features. Examples of additional nucleic acid features that can be included in a viral vector provided herein include, without limitation, origins of replication, nucleic acid encoding a selectable marker, nucleic acid encoding a detectable label (e.g. Flag, 3×Flag, myc, and V5), miRNA target sequences, and ASO targeting sequences.

In some cases, expression of a truncated Syngap1 polypeptide (e.g., a Syngap1-B:α1 polypeptide) from a viral vector provided herein (e.g., a viral vector that includes nucleic acid encoding a truncated Syngap1 polypeptide such as a Syngap1-B:α1 polypeptide) can be regulated. For example, one or more nucleic acid molecules that can (e.g., that are designed to) bind a nucleic acid sequence present in a viral vector provided herein (e.g., but not present in an endogenous SYNGAP1 gene) can reduce or eliminate expression of a truncated Syngap1 polypeptide (e.g., a Syngap1-B:α1 polypeptide) from that viral vector. In some cases, a nucleic acid molecule can that bind a nucleic acid sequence present in a viral vector provided herein can be an ASO. Examples of nucleic acid molecules that can bind a nucleic acid sequence present in a viral vector provided herein (e.g., but not present in an endogenous SYNGAP1 gene) include, without limitation, nucleic acid molecules having a nucleic acid sequence set forth in any one of SEQ ID NOs:12-21 (see, e.g., Table 1).

This document also provides compositions containing one or more viral vectors (e.g., AAV vectors) provided herein (e.g., a viral vector that includes nucleic acid encoding a truncated Syngap1 polypeptide such as a Syngap1-B:α1 polypeptide). For example, one or more viral vectors provided herein can be formulated into a composition (e.g., a pharmaceutically acceptable composition) for administration to a mammal (e.g., a human) having, or at risk for developing, a SYNGAP1-associated NDD (e.g., SRID). For example, one or more one or more viral vectors (e.g., AAV vectors) provided herein (e.g., a viral vector that includes nucleic acid encoding a truncated Syngap1 polypeptide such as a Syngap1-B:α1 polypeptide) can be formulated together with one or more pharmaceutically acceptable carriers (additives) and/or diluents. Pharmaceutically acceptable carriers, fillers, and vehicles that may be used in a pharmaceutical composition described herein include, without limitation, saline, dimethyl sulfoxide (DMSO), ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat.

A composition including one or more viral vectors (e.g., AAV vectors) provided herein (e.g., a viral vector that includes nucleic acid encoding a truncated Syngap1 polypeptide such as a Syngap1-B:α1 polypeptide) can be designed for oral or parenteral (including ICV, intracerebral, intracerebellar, intrathecal, sinus, retroorbital, and intravenous) administration to a mammal (e.g., a human) having, or at risk for developing, a SYNGAP1-associated NDD (e.g., SRID). Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition including one or more viral vectors (e.g., AAV vectors) provided herein (e.g., a viral vector that includes nucleic acid encoding a Syngap1-B:α1 polypeptide) can be formulated for parenteral administration (e.g., ICV injection, intravenous (IV) injection, and intracranial (IC) injection).

In some cases, a composition containing one or more viral vectors (e.g., AAV vectors) provided herein (e.g., a viral vector that includes nucleic acid encoding a truncated Syngap1 polypeptide such as a Syngap1-B:α1 polypeptide) can be in the form of a sterile injectable suspension (e.g., a sterile injectable aqueous or oleaginous suspension). This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1,3-butanediol. Examples of acceptable vehicles and solvents that can be used include, without limitation, saline, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides.

In some cases, a composition containing one or more (e.g., one, two, three, four, or more) lipase inhibitors can be presented in unit-dose or multi-dose containers, for example, sealed ampules and vials, and may be stored in a freeze dried (lyophilized) condition requiring only the addition of the sterile liquid carrier, for example water for injections, immediately prior to use.

A composition including one or more viral vectors (e.g., AAV vectors) provided herein (e.g., a viral vector that includes nucleic acid encoding a truncated Syngap1 polypeptide such as a Syngap1-B:α1 polypeptide) can be designed for any type of release (e.g., release of the one or more viral vectors provided herein from the composition) into the mammal that the composition is administered to (e.g., a mammal having, or at risk for developing, a SYNGAP1-associated NDD such as SRID). For example, a composition including one or more viral vectors (e.g., AAV vectors) provided herein (e.g., a viral vector that includes nucleic acid encoding a truncated Syngap1 polypeptide such as a Syngap1-B:α1 polypeptide) can be designed for immediate release, slow release, or extended release of the one or more viral vectors provided herein.

This document also provides methods and materials for using one or more viral vectors (e.g., AAV vectors) provided herein (e.g., a viral vector that includes nucleic acid encoding a truncated Syngap1 polypeptide such as a Syngap1-B:α1 polypeptide). For example, one or more viral vectors provided herein can be administered to a mammal (e.g., a human) having, or at risk for developing, a SYNGAP1-associated NDD such as SRID (e.g., to treat the mammal). For example, one or more viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) to increase the level of one or more Syngap1 polypeptides (e.g., one or more truncated Syngap1 polypeptides such as Syngap1-B:α1 polypeptides) within neurons within the mammal.

Any type of mammal having, or at risk for developing, a SYNGAP1-associated NDD (e.g., SRID) can be treated as described herein. In some cases, a mammal to be treated as described herein can be an infant (e.g., a newborn). In some cases, a mammal to be treated as described herein can be in utero. Examples of mammals that can have, or can be at risk for developing, a SYNGAP1-associated NDD (e.g., SRID) and can be treated as described herein include, without limitation, humans, non-human primates such as monkeys, dogs, cats, horses, cows, pigs, sheep, rabbits, mice, and rats.

In some cases, methods described herein can include identifying a mammal (e.g., a human) as having or being at risk of developing a SYNGAP1-associated NDD (e.g., SRID).

Any appropriate method can be used to identify a mammal as having, or at risk for developing, a SYNGAP1-associated NDD (e.g., SRID). For example, genetic testing can be used to identify a human or other mammal as having, or at risk for developing, a SYNGAP1-associated NDD (e.g., SRID).

When treating a SYNGAP1-associated NDD as described herein, the SYNGAP1-associated NDD can be any appropriate SYNGAP1-associated NDD. Examples of SYNGAP1-associated NDD that can be treated as described herein (e.g., by administering one or more viral vectors provided herein such as an AAV vector that includes nucleic acid encoding a truncated Syngap1 polypeptide such as a Syngap1-B:α1 polypeptide) include, without limitation, SRID, SYNGAP1-related autism spectrum disorder (ASD), SYNGAP1-related epilepsy, sleep disorders, and schizophrenia.

A mammal (e.g., a human) having, or at risk for developing, a SYNGAP1-associated NDD (e.g., SRID) can be administered or instructed to self-administer any one or more viral vectors (e.g., AAV vectors) provided herein (e.g., a viral vector that includes nucleic acid encoding a truncated Syngap1 polypeptide such as a Syngap1-B:α1 polypeptide).

In some cases, one or more viral vectors (e.g., AAV vectors) provided herein (e.g., a viral vector that includes nucleic acid encoding a truncated Syngap1 polypeptide such as a Syngap1-B:α1 polypeptide) can be used to increase the level of one or more Syngap1 polypeptides (e.g., one or more truncated Syngap1 polypeptides such as Syngap1-B:α1 polypeptides) within neurons within a mammal (e.g., a human). For example, one or more viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) in need thereof (e.g., a human having, or at risk for developing, a SYNGAP1-associated NDD such as SRID) to increase the level of one or more Syngap1 polypeptides (e.g., one or more truncated Syngap1 polypeptides such as Syngap1-B:α1 polypeptides) within neurons within the mammal. The term “increased level” as used herein with respect to a level of a truncated Syngap1 polypeptide (e.g., a Syngap1-B:α1 polypeptide) in a mammal having, or at risk for developing, a SYNGAP1-associated NDD (e.g., SRID) refers to any level that is greater than the level of that Syngap1 polypeptide observed in that mammal prior to being treated as described herein. In some cases, an increased level of a Syngap1 polypeptide can be a level that is at least 1.2 fold (e.g., about 1.3 fold, about 2 fold, about 3, fold, about 5 fold, about 7 fold, about 10 fold, about 12 fold, about 15 fold, about 17 fold, about 20 fold, or more) higher than the level of that Syngap1 polypeptide prior to being treated as described herein. In some cases, an increased level of a Syngap1 polypeptide can be a level that from about 1.2 fold to about 20 fold higher than the level of that Syngap1 polypeptide prior to being treated as described herein. In some cases, an increased level of a Syngap1 polypeptide can be a level that from about 1.3 fold to about 20 fold higher than the level of that Syngap1 polypeptide prior to being treated as described herein. In some cases, an increased level of a Syngap1 polypeptide can be a level that is at least 5 percent (e.g., at least 10, at least 15, at least 20, at least 25, at least 35, at least 50, at least 75, at least 100, or at least 150 percent) higher than the level of that Syngap1 polypeptide prior to being treated as described herein. In some cases, when samples have an undetectable level of a Syngap1 polypeptide prior to treatment as described herein, an increased level can be any detectable level of a Syngap1 polypeptide. It will be appreciated that levels from comparable samples are used when determining whether or not a particular level is an increased level.

In cases where one or more viral vectors (e.g., AAV vectors) provided herein (e.g., a viral vector that includes nucleic acid encoding a truncated Syngap1 polypeptide such as a Syngap1-B:α1 polypeptide) are used to increase the level of one or more Syngap1 polypeptides (e.g., one or more truncated Syngap1 polypeptides such as Syngap1-B:α1 polypeptides) within neurons within a mammal (e.g., a human), and the one or more viral vectors include a nucleic acid sequence that can be targeted by one or more nucleic acid molecules (e.g., one or more ASOs), the expression level of the one or more Syngap1 polypeptides can be regulated by also administering the one or more nucleic acid molecules (e.g., the one or more ASOs) to the mammal. One or more nucleic acid molecules (e.g., one or more ASOs) that can be used to regulate one or more vectors provided herein can be administered to a mammal (e.g., a human) in any appropriate form. Examples of nucleic acid molecules that can bind a nucleic acid sequence present in a viral vector provided herein (e.g., but not present in an endogenous SYNGAP1 gene) and can be used to regulate a level of one or more Syngap1 polypeptides expressed by a viral vector provided herein include, without limitation, nucleic acid molecules having a nucleic acid sequence set forth in any one of SEQ ID NOs:12-21 (see, e.g., Table 1).

In some cases, one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) can be used to reduce the severity of one or more symptoms of a SYNGAP1-associated NDD (e.g., SRID). For example, one or more lipase inhibitors can be administered to a mammal (e.g., a human) in need thereof (e.g., a human having or at risk of developing a SYNGAP1-associated NDD (e.g., SRID)) to reduce the severity of one or more symptoms of the SYNGAP1-associated NDD (e.g., SRID). Examples of symptoms of a SYNGAP1-associated NDD (e.g., SRID) include, without limitation, gross motor delays (e.g., in infancy), developmental delays, seizures, language impairment, and sleep disorders. In some cases, the methods and materials described herein can be effective to reduce the severity of one or more symptoms of a SYNGAP1-associated NDD (e.g., SRID) in a mammal having SYNGAP1-associated NDD (e.g., SRID) by, for example, 10, 20, 30, 40, 50, 60, 70, 80, 90, 95, or more percent.

In some cases, one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) can be used to reduce or slow the progression of a SYNGAP1-associated NDD (e.g., SRID). For example, one or more viral vectors provided herein can be administered to a mammal (e.g., a human) in need thereof (e.g., a human having a SYNGAP1-associated NDD such as SRID) to reduce or slow the progression of a SYNGAP1-associated NDD (e.g., SRID) in the mammal. In some cases, the methods and materials described herein can be effective to reduce or slow the progression of a SYNGAP1-associated NDD (e.g., SRID) in a mammal having a SYNGAP1-associated NDD (e.g., SRID) by, for example, 10, 20, 30, 40, 50, 60, 70, 80, 90, 95, or more percent. In some cases, the methods and materials described herein can be effective to reduce or slow the progression of a SYNGAP1-associated NDD (e.g., SRID) in a mammal having a SYNGAP1-associated NDD (e.g., SRID) by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) can be used to delay or prevent the development of a SYNGAP1-associated NDD (e.g., SRID). For example, one or more viral vectors provided herein can be administered to a mammal (e.g., a human) in need thereof (e.g., a human at risk of developing a SYNGAP1-associated NDD such as SRID) to delay or prevent the development of a SYNGAP1-associated NDD (e.g., SRID) in the mammal. In some cases, the methods and materials described herein can be effective to delay the development of a SYNGAP1-associated NDD (e.g., SRID) in a mammal at risk of developing a SYNGAP1-associated NDD (e.g., SRID) by, for example, 10, 20, 30, 40, 50, 60, 70, 80, 90, 95, or more percent. In some cases, the methods and materials described herein can be effective to delay the development of a SYNGAP1-associated NDD (e.g., SRID) in a mammal at risk of developing a SYNGAP1-associated NDD (e.g., SRID) by, for example, at least 6 months (e.g., about 6 months, about 8 months, about 10 months, about 1 year, about 1.5 years, about 2 years, about 2.5 years, about 3 years, about 4 years, about 5 years, or more).

A composition containing one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) in any appropriate amount (e.g., any appropriate dose). An effective amount of a composition containing one or more viral vectors provided herein can be any amount that can treat a mammal having, or at risk for developing, a SYNGAP1-associated NDD (e.g., SRID) as described herein without producing significant toxicity to the mammal. For example, an effective amount of the viral vector(s) provided herein can be from about 1.0×10¹¹ viral genomes per kg body weight of the mammal to be treated (vg/kg) to about 1.0×10¹⁷vg/kg. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the SYNGAP1-associated NDD (e.g., SRID) in the mammal being treated may require an increase or decrease in the actual effective amount administered.

A composition containing one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) in any appropriate frequency. The frequency of administration can be any frequency that can treat a mammal having, or at risk for developing, a SYNGAP1-associated NDD (e.g., SRID) without producing significant toxicity to the mammal. For example, the frequency of administration can be from about twice a day to about one every other day, once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) can be administered to a mammal (e.g., a human) having, or at risk for developing, a SYNGAP1-associated NDD (e.g., SRID) for any appropriate duration. An effective duration for administering or using a composition containing one or more viral vectors provided herein can be any duration that can treat a mammal having, or at risk for developing, a SYNGAP1-associated NDD (e.g., SRID) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having, or at risk for developing, a SYNGAP1-associated NDD (e.g., SRID) as described herein (e.g., by administering one or more viral vectors provided herein such as an AAV vector that includes nucleic acid encoding a truncated Syngap1 polypeptide such as a Syngap1-B:α1 polypeptide) can include administering to the mammal one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) as the sole active ingredient to treat the mammal. For example, a composition containing one or more viral vectors provided herein can include the one or more viral vectors as the sole active ingredient in the composition that is effective to treat a mammal having, or at risk for developing, a SYNGAP1-associated NDD (e.g., SRID).

In some cases, methods for treating a mammal (e.g., a human) having, or at risk for developing, a SYNGAP1-associated NDD (e.g., SRID) as described herein (e.g., by administering one or more viral vectors provided herein such as an AAV vector that includes nucleic acid encoding a truncated Syngap1 polypeptide such as a Syngap1-B:α1 polypeptide) also can include administering to the mammal one or more (e.g., one, two, three, four, five or more) additional agents used to treat SYNGAP1-associated NDD (e.g., SRID) to the mammal and/or performing therapies used to treat SYNGAP1-associated NDD (e.g., SRID) on the mammal. For example, a combination therapy used to treat SYNGAP1-associated NDD (e.g., SRID) can include administering to the mammal (e.g., a human) one or more viral vectors (e.g., AAV vectors) provided herein (e.g., a viral vector that includes nucleic acid encoding a truncated Syngap1 polypeptide such as a Syngap1-B:α1 polypeptide) and one or more (e.g., one, two, three, four, five or more) agents used to treat SYNGAP1-associated NDD (e.g., SRID). Examples of agents that can be administered to a mammal to treat SYNGAP1-associated NDD (e.g., SRID) include, without limitation, anti-seizure agents, antipsychotic agents (e.g., risperdal), ADHD treatments (e.g., guanfacine), sleep disorder treatments, and any combinations thereof. In cases where one or more viral vectors provided herein are used in combination with additional agents used to treat SYNGAP1-associated NDD (e.g., SRID), the one or more additional agents can be administered at the same time (e.g., in a single composition containing both one or more viral vectors provided herein and the one or more additional agents) or independently. For example, one or more viral vectors provided herein can be administered first, and the one or more additional agents administered second, or vice versa.

In some cases, a combination therapy used to treat SYNGAP1-associated NDD (e.g., SRID) can include administering to the mammal (e.g., a human) one or more (e.g., one, two, three, four, or more) viral vectors (e.g., AAV vectors) provided herein (e.g., a viral vector that includes nucleic acid encoding a truncated Syngap1 polypeptide such as a Syngap1-B:α1 polypeptide) and performing one or more (e.g., one, two, three, four, five or more) additional therapies used to treat SYNGAP1-associated NDD (e.g., SRID) on the mammal. Examples of therapies used to treat SYNGAP1-associated NDD (e.g., SRID) include, without limitation, occupational therapy, physical therapy, speech and language therapy, applied behavioral analysis therapy, and/or developmental therapy. In cases where one or more viral vectors provided herein are used in combination with one or more additional therapies used to treat SYNGAP1-associated NDD (e.g., SRID), the one or more additional therapies can be performed at the same time or independently of the administration of one or more viral vectors provided herein. For example, one or more viral vectors provided herein can be administered before, during, or after the one or more additional therapies are performed.

In certain instances, a course of treatment and the severity of one or more symptoms related to the condition being treated (e.g., a SYNGAP1-associated NDD such as SRID) can be monitored. Any appropriate method can be used to determine whether or not the severity of a symptom is reduced. For example, the severity of a symptom of a SYNGAP1-associated NDD (e.g., SRID) can be assessed using EEG, epilepsy measurements, hyperactivity, working memory behavior, biomarker assays, sleep measurements, and/or behavioral assessments at different time points.

The invention will be further described in the following examples, which do not limit the scope of the invention described in the claims.

### EXAMPLES

### Example 1: Therapeutic SYNGAP1 AAV Vector

This Example describes a Syngap1-B:α1 polypeptide that can be used to treat SYNGAP1 haploinsufficiency. For example, a nucleic acid encoding the Syngap1-B:α1 polypeptide can be packaged into an AAV vector and for AAV-mediated gene therapy.

### Characterization of AAV-SYNGAP1 Vector

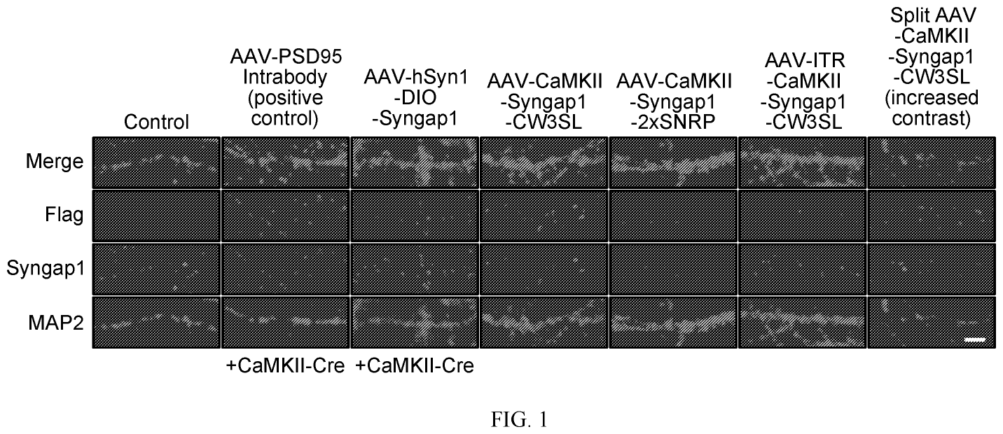

An AAV1-SYNGAP1 vector was designed to drive robust expression of a Syngap1-B:α1 polypeptide in excitatory neurons using a short CaMKIIα promoter (0.4 kb) and optimized ITR/3′UTR elements. Immature neurons (DIV5-6) were treated for 10-20 days. Immunocytochemistry was used to confirm proper dendritic spine localization of the exogenously expressed Syngap1-B:α1 polypeptide (FIG. 1).

### Expression of AAV-SYNGAP1 Vector

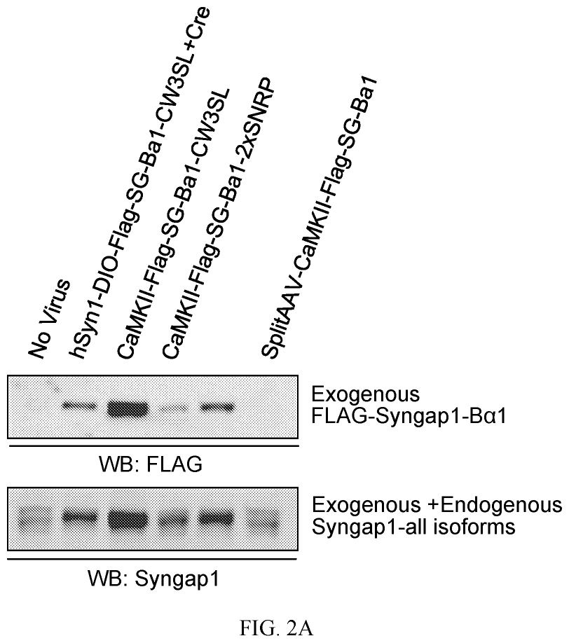

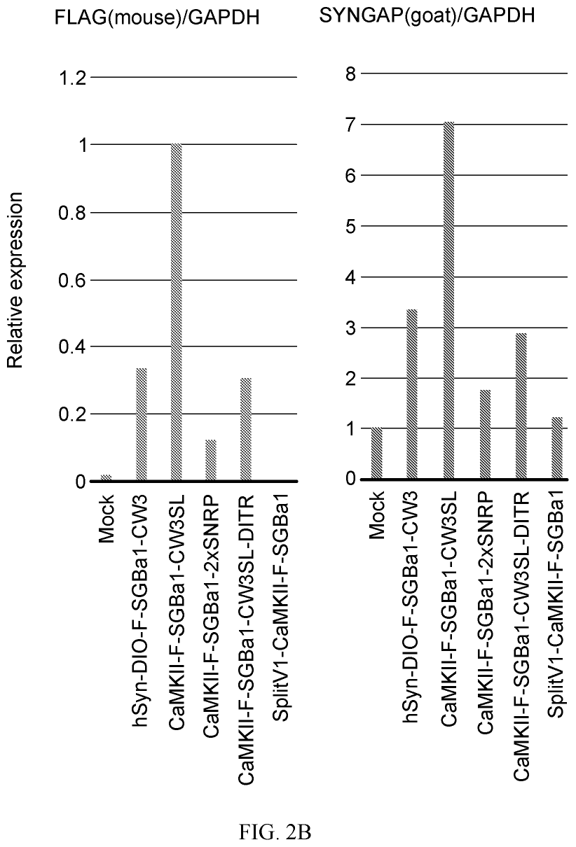

To screen AAV-SYNGAP1 variants in vitro, primary cultured neurons were treated with an AAV1-SYNGAP1 vector, and expression of a Syngap1-B:α1 polypeptide was evaluated with Western blots. Results demonstrated a ˜7-fold higher expression of a Syngap1-B:α1 polypeptide compared to no virus controls for the AAV-CaMKIIα-Syngap1-CW3SL variant (FIGS. 2A-2B).

### Anti-Sense Oligonucleotide (ASO) ‘Kill-Switches’ for AAV-SYNGAP1 Vector

The persistent nature of AAV treatment necessitates robust safety measures especially in non-dividing cells like neurons and with genes that may cause functional impairment with overexpression like MECP2. The effect of SYNGAP1 overexpression is currently unknown, so it will be critical to establish the safety of any gene therapy candidate treatment. To provide means to pre-mitigate this safety issue, an ASO (antisense oligonucleotide)-based strategy to allow specific modulation of the gene therapy vector through uniquely targeted ASOs that do not engage the endogenous gene were devised and tested. For patients that require precise expression control, these ASOs provide critical ‘kill-switches’ for regulatable gene therapy.

A series of ASOs were designed to allow specific modulation of the AAV-SYNGAP1 vectors through uniquely targeted ASOs that do not engage the endogenous SYNGAP1 gene (Table 1). These ASOs target the Flag tag sequence, the junction between Flag and SYNGAP1, and codon-optimized regions of the SYNGAP1 cDNA for specificity.

TABLE 1
Kill-switch ASOs designed to target
specific AAV-Syngap1 isoforms.

| Target | ASO Name | ASO Sequence | SEQ ID NO: |
|---|---|---|---|
| all Syngap1 AAVs | ASO-10 | CCTTATCGTCATCGTCCTTG | 12 |
| just AAV-SYNGAP1-Bα2 | ASO-111 | TTACGGAATTCACCGTTCTC | 13 |
| just AAV-SYNGAP1-Bα1 | ASO-17 | TAAGGCCCTTATCGTCATCG | 14 |
| just AAV-SYNGAP1-Bα2 | ASO-17-5 | AGACCTTTATCGTCATCGTC | 15 |
| just AAV-SYNGAP1-Bβ | ASO-17-6 | AATCCTTTATCGTCATCGTC | 16 |
| just AAV-SYNGAP1-Bγ | ASO-17-7 | AACCCTTTATCGTCATCGTC | 17 |
| just AAV-SYNGAP1-Bα1 | ASO-4-1 | TATCTCCTCTTCGTACTCTT | 18 |
| just AAV-SYNGAP1-Bα2 | ASO-4-2 | AATCTCCTCTTCGTATTCCT | 19 |
| just AAV-SYNGAP1-Bβ | ASO-4-3 | GATCTCTTCTTCGTACTCCT | 20 |
| just AAV-SYNGAP1-Bγ | ASO-4-4 | GATTTCCTCTTCGTACTCCT | 21 |
| Sum | 10 ASOs | | |

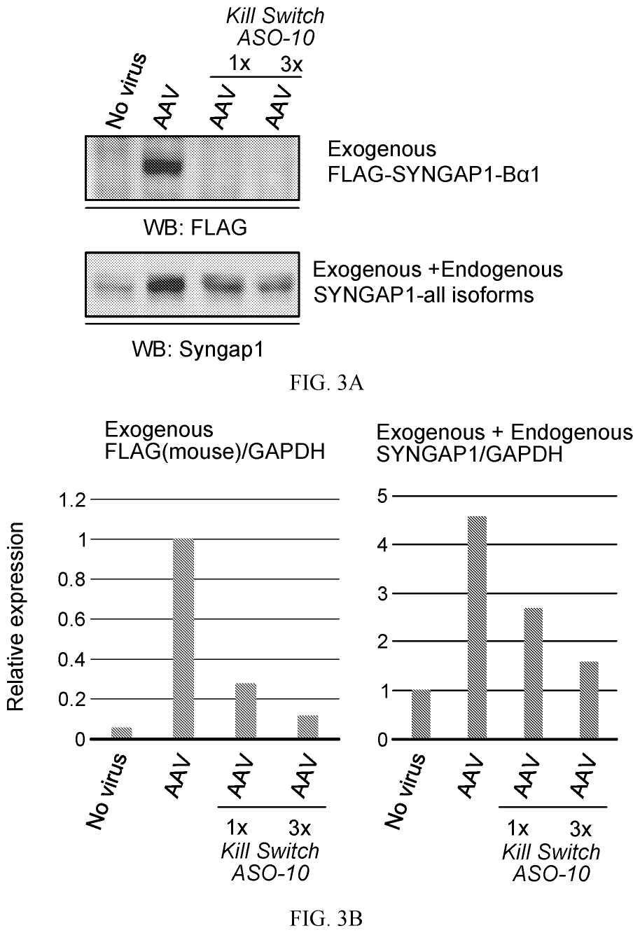

These ASOs demonstrate a 7-20 fold suppression of AAV-mediated SYNGAP 1 expression in a dose-dependent fashion (FIGS. 3A-3B).

Expression of Alternative c-Terminal Isoforms (α1/α2/β/γ) with AAV-SYNGAP1 Vector

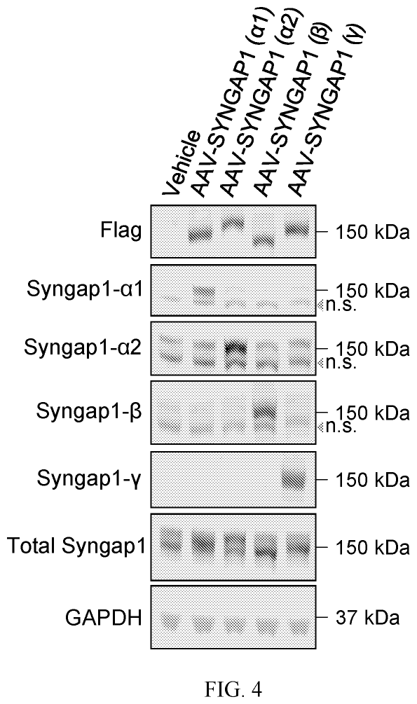

To enable the expression of various c-terminal isoforms with AAV-SYNGAP1, AAVs with the coding sequence of the α2/β/γ isoforms were generated. These variants were tested in vitro on primary cultured neurons, and expression of Flag-Syngap1-B:α1/α2/P/γ polypeptides was evaluated with Western blots. Results demonstrated a robust expression of all isoforms (FIG. 4). These AAVs allowed the expression of all significant c-terminal isoforms (α3 is only one amino acid different from α2 and is a minor isoform). Isoforms can be mixed (e.g., at native ratios) to achieve a similar mixture of expressed isoforms as observed in the normal brain (n.s. denotes non-specific bands).

### Capsid Optimization of the AAV-SYNGAP1 Vector

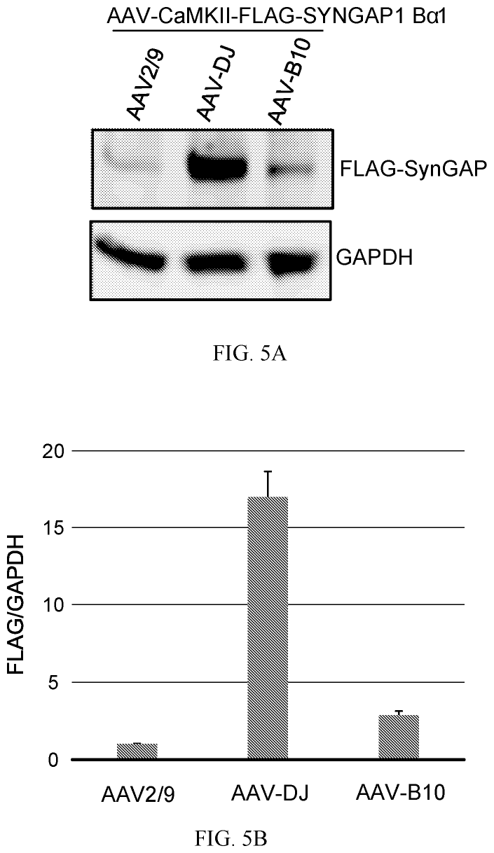

To optimize the efficient infection and expression of AAV-SYNGAP1, AAVs with alternative capsids AAV-DJ and AAV-B10 were generated, in addition to the previously described AAV9s. These capsids provide increased tropism and BBB penetration. These variants were tested in vitro on primary cultured neurons, and expression of the Flag-Syngap1-B:α1 polypeptide was evaluated with Western blots. Results demonstrated stronger expression from the AAV-DJ and AAV-B10 capsid variants (FIG. 5). This suggests that AAV-SYNGAP1 capsid optimization can afford more efficient and broad expression in neurons and patients.

scAAV-Adaptable Truncated SYNGAP1 Rescues of SYNGAP1 KD Spine Phenotype

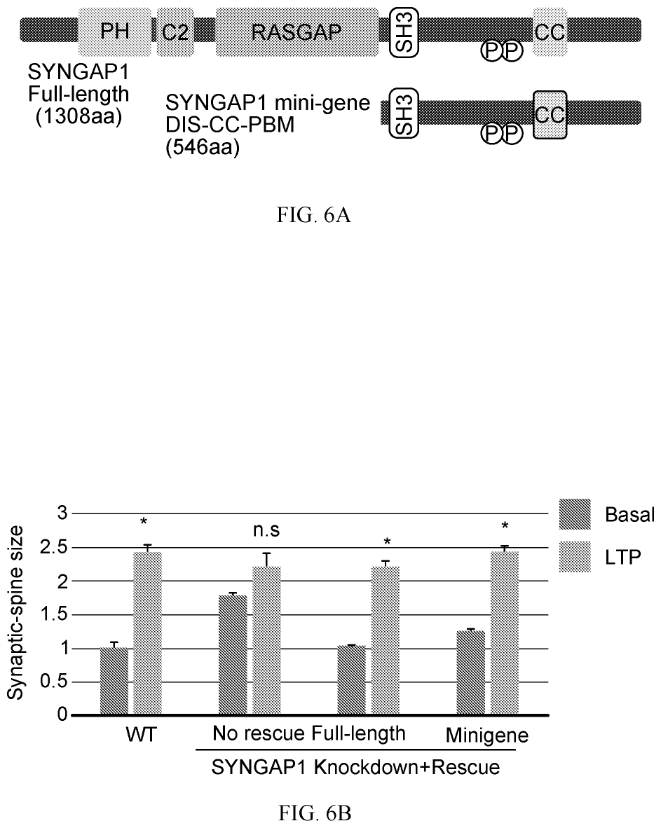

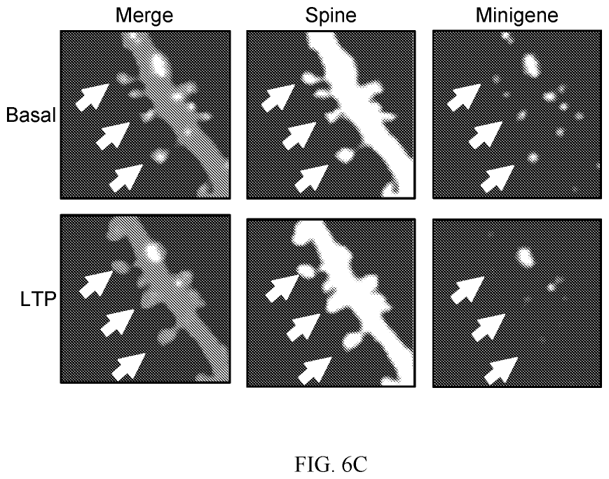

scAAV vectors provide accelerated infection and expression of gene therapy cargo. Through further truncation screens, it was discovered that a radically truncated c-terminal fragment of SynGAP1 rescued KD-induced spine deficits (FIGS. 6A-6C). This truncated form spans only 1.6 kb, making it amenable to scAAV vector technology.

scAAV-SYNGAP1 and Codon-Optimized ssAAV

An Exon 14-20 minigene SYNGAP1 was packaged into scAAV with 3×Flag and two different poly-A signals (CW3SL and bGHpA). The CW3SL version is slightly oversized (2701 bp), whereas the bGHpA version (2499 bp) is within the theoretical scAAV cargo capacity (2.5kb). Compared to the ssAAV (single-strand AAV) version of AAV-SYNGAP (AAV-CaMKII-Flag-hSYNGAP-B:α1-CW3SL V2), the scAAV versions both expressed more SYNGAP1, calculated as the sum of the 65 and 150 kDa bands. The bGHpA version of scAAV-SYNGAP1 expressed roughly 4-fold of the ssAAV-SYNGAP1.

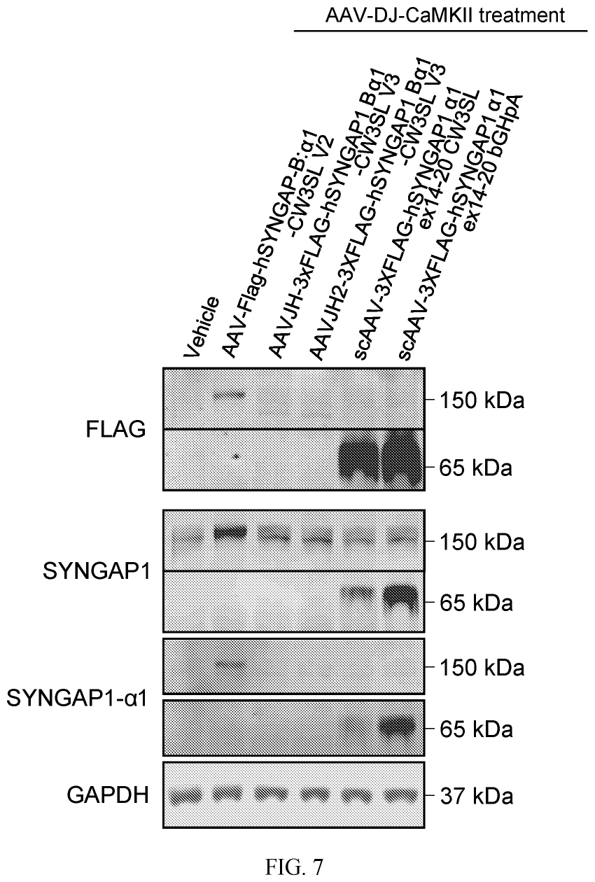

AAV-SYNGAP1 vectors were optimized for safety (see, e.g., Hamilton et al., Front. Immunol., 12:675897 (2021); Zanta-Boussif et al., Gene Ther., 16(5):605-19 (2009); Richter et al., Cell, 163(2):292-300 (2015); and Wu et al., eLife, 8:e45396 (2019)), and a third generation of AAVs (V3) was derived and tested (FIG. 7). These V3 vectors provide a basis for safe and efficient administration.

Together, these results demonstrate that AAV vectors (e.g., scAAV vectors) can be used to express a Syngap1 polypeptide (e.g., Syngap1-B:α1 polypeptide) in neurons, and can therefore be used to treat a disease or disorder associated with SYNGAP1 haploinsufficiency such as a SYNGAP1-associated NDD. This is a significant step towards efficient gene therapy, enabled by the discovery of a functional SYNGAP1 truncations.

### Example 2: Examples of Syngap1 Sequences

TABLE 2
Examples of Nucleic Acid Sequences that can encode a Syngap1 polypeptides

| Sequence Type | SEQ ID NO |
|---|---|
| Human wild-type SYNGAP1-B:α1 coding sequence | 1 |
| Codon-optimized SYNGAP1-B:α1 coding sequence | 2 |
| Codon-optimized SYNGAP1-B:α2 coding sequence | 3 |
| Codon-optimized SYNGAP1-B:β coding sequence | 4 |
| Codon-optimized SYNGAP1-B:γ coding sequence | 5 |
| Flag-SYNGAP1-B:α1 | 6 |
| Codon-optimized and CpG replaced SYNGAP1-B:α1 coding sequence | 22 |

TABLE 3
Examples of Syngap1 polypeptides.

| Syngap1 polypeptide | SEQ ID NO |
|---|---|
| Syngap1-B:α1 | 7 |
| SYNGAP1-B:α2 | 8 |
| SYNGAPI-B:β | 9 |
| SYNGAPI-B:γ | 10 |
| N-terminal truncated SYNGAP1-α1 | 11 |
| N-terminal truncated SYNGAP1-α1 (exon 14-20) | 23 |
| N-terminal truncated SYNGAP1-α2 (exon 14-20) | 24 |
| N-terminal truncated SYNGAP1-β (exon 14-20) | 25 |
| N-terminal truncated SYNGAP1-γ (exon 14-20) | 26 |

### Example 3: Treating a SYNGAP1-Associated NDD

A human identified as having SYNGAP1-associated NDD (e.g., SRID) is administered (e.g., by ICV injection) a population of AAV vectors including nucleic acid encoding a Syngap1-B:α1 polypeptide. The administered AAV vectors increase the level of the Syngap1-B:α1 polypeptide within neurons in the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A viral vector comprising a nucleic acid encoding a truncated Syngap1 polypeptide.

2. The viral vector of claim 1, wherein said viral vector is selected from the group consisting of an adeno-associated viral (AAV) vector, a retroviral vector, a rhabdovirus-based vector, an adenovirus vector, and a herpes simplex virus vector.

3. The viral vector of claim 2, wherein said viral vector is an AAV vector.

4. The viral vector of claim 1, wherein said truncated Syngap1 polypeptide is selected from the group consisting of a Syngap1-B:α1 polypeptide, a Syngap1-B:α2 polypeptide, a Syngap1-B:α3 polypeptide, a Syngap1-B:β polypeptide, a Syngap1-B:γ polypeptide, a Syngap1-α1 polypeptide, a Syngap1-α2 polypeptide, a Syngap1-α3 polypeptide, a Syngap1-p polypeptide, a Syngap1-γ polypeptide, and a Syngap1-exon14-20 polypeptide.

5. The viral vector of claim 1, wherein said truncated Syngap1 polypeptide comprises an amino acid sequence set forth in any one of SEQ ID NOs:7-11 or SEQ ID NOs:23-26.

6. The viral vector of claim 1, wherein said nucleic acid encoding said truncated Syngap1 polypeptide comprises a nucleic acid sequence set forth in any one of SEQ ID NOs:1-6 or SEO ID NO:22.

7. The viral vector of claim 1, wherein said nucleic acid encoding said truncated Syngap1 polypeptide is operably linked to a promoter.

8. The viral vector of claim 7, wherein said promoter is a neuron-specific promoter.

9. The viral vector of claim 7, wherein the promoter is selected from the group consisting of a CaMKIIα promoter, a SYN1 promoter, and a SYNGAP1 promoter.

10. The viral vector of claim 1, wherein said viral vector comprises an optimized inverted terminal repeat (ITR).

11. The viral vector of claim 1, wherein said viral vector comprises an optimized 3′ untranslated region (UTR).

12. The viral vector of claim 11, where said optimized 3′ UTR is selected from the group consisting of a SV40, hGH, BGH, rbGlob, CW3SL, and 2×SNRP.

13. The viral vector of claim 1, wherein said viral vector comprises a nucleic acid sequence that can targeted by an anti-sense oligonucleotide (ASO).

14-15. (canceled)

16. The viral vector of claim 13, wherein said ASO comprises a sequence set forth in any one of SEQ ID NOs:12-21.

17. A method for treating a mammal having or at risk of developing a SYNGAP1-associated neurodevelopmental disorder (NDD), wherein said method comprises:
administering to said mammal a viral vector of claim 1.

18. The method of claim 17, wherein said mammal is a human.

19-20. (canceled)

21. The method of claim 17, wherein said SYNGAP1-associated NDD is selected from the group consisting of a SYNGAP1-related intellectual disability (SRID), SYNGAP1-related autism spectrum disorder (ASD), SYNGAP1-related epilepsy, a sleep disorder, and schizophrenia.

22. The method of claim 17, wherein said administering comprises an intracerebroventricular (ICV) injection.

23. A method for increasing a level of a truncated Syngap1 polypeptide within neurons of a mammal, wherein said method comprises:
administering to said mammal a viral vector of claim 1.

24. The method of claim 23, wherein said mammal is a human.

25-34. (canceled)

35. The viral vector of claim 3, wherein said truncated Syngap1 polypeptide is said Syngap1-exon14-20 polypeptide.

36. The viral vector of claim 35, wherein said Syngap1-exon14-20 polypeptide is selected from the group consisting of a Syngap1-exon14-20al polypeptide, a Syngap1-exon14-20:α2 polypeptide, a Syngap1-exon14-20:α3 polypeptide, a Syngap1-exon14-20:0 polypeptide, and a Syngap1-exon14-20:Y polypeptide.

37-38. (canceled)

39. The viral vector of any one of claim 3, wherein said AAV vector is selected from the group consisting of an AAV2 vector, an AAV2/9 vector, an AAV9 vector, an AAV1 vector, an AAVrh10 vector, an AAV.B10 vector, and an AAV-DJ vector.

40. The viral vector of claim 1, wherein said viral vector is a self-complementary AAV (scAAV) vector.
